(19) United States
(12) Patent Application Publication
(10) Pub. No.: US 2005/0175143 A1
(43) Pub. Date: Aug. 11, 2005

(54) Multi-slice x-ray ct device

(76) Inventors: Osamu Miyazaki, Moriya (JP); Taiga Goto, Kashiwa (JP); Hiroto Kokubun, Kashiwa (JP)

Correspondence Address: COOPER & DUNHAM, LLP, 1185 AVENUE OF THE AMERICAS, NEW YORK, NY, 10036, US

(21) Appl. No.: 10/515,289

(22) PCT Filed: Jun. 3, 2003
(86) PCT No.: PCT/JP03/07008

(30) Foreign Application Priority Data
Jun. 3, 2002 (JP) 2002-160940
Jun. 3, 2002 (JP) 0220-160952

(51) Int. Cl. G21K 1/12; A61B 6/00

(52) U.S. Cl. 378/19

(57) ABSTRACT

Three pairs of X-ray tubes (21A-21C) and single- or multiple-row detectors (31A-31C) are mounted on a rotary disc (49) installed in a scanner unit (12) at a rotational phase difference of 120°, and a deviation (offset) ΔZ is set between the three pairs in a rotation axis direction of a subject (16) in accordance with ΔZ=d×N, where d is the thickness of the row of the single- or multiple-row detectors (31A-31C), and N is an offset coefficient. Slice collimators (48A-48C) are provided to X-ray tubes (21A-21C) in the three pairs, and are rotated relative to the subject (16) to provide a high-quality tomographic image with high temporal resolution, less motion artifact and high space resolution.

### TECHNICAL FIELD

The present invention relates to an X-ray CT (Computed Tomography) device for capturing a tomographic image of a subject.

### BACKGROUND ART

Since the X-ray CT device was developed, attempts have been consistently made to reduce a testing time till recent years.

FIG. 35 illustrates a schematic diagram of an X-ray CT device. The X-ray CT device comprises a host computer 11 for totally controlling the whole system; a scanner 12 having X-ray tubes, detectors, and a rotary scanning mechanism mounted with a rotary disc; and a high voltage generator 5 which is a power source of the X-ray tube. The X-ray CT device also comprises a subject table 13 for carrying a subject 16 when the subject 16 is positioned and during a helical scan; an image processing unit 14 for performing a variety of image processing, represented by pre-processing and reconstruction processing; and a display device 17 for displaying a tomographic image of the subject 16.

For the detectors, a single-row detector based X-ray computer tomographic imaging device, which employs a single row of detectors, determines the thickness of a slice for tomographic imaging by collimating (limiting) the slice to an arbitrary width by a slice collimator before the subject is irradiated with X-rays.

On the other hand, a multi-row detector based X-ray computer tomographic imaging device (MDCT: Multi Detector CT), which has a plurality of detector rows in a rotation axis direction, the thickness of a slice is determined by the width of elements of the detectors in the rotation axis direction.

A means for realizing a higher speed in such a mechanical scan based X-ray CT device may utilize a plurality of X-ray tubes (multi-tubes). Among others, an X-ray CT device which has a structure comprised of three single-row detectors in the rotation axis direction corresponding to the respective X-ray tubes has been disclosed as an invention of the third generation system in JP-A-54-152489 which describes that the X-ray tubes can be independently moved in the rotation axis direction. In this third generation system, techniques have also been disclosed for shifting pairs of an X-ray tube and a detector row in a rotational axis direction for scanning in such a manner that the same helical (spiral) trajectory is achieved (see JP-A-06-038957).

(Problem to be Solved by the Invention)

However, the mechanical scanning CT device using a single-row detector is thought to be limited in a rotating time of one rotation to approximately 0.3-0.4 seconds in consideration of the anti-vibration performance of a rotary anode X-ray tube. Also, a maximally allowed load is thought to be limited to approximately 500 mA of a tube current of the X-ray tube. For a 0.3-second scan, the tube current of the X-ray tube is calculated by 0.3×500=150 mAs, giving rise to a problem of a failure in ensuring a sufficient X-ray dose. While this X-ray CT device employing rotary anode X-ray tube is capable of applying a maximum tube current of 700 mA, the problem of an insufficient dose still remains unsolved for imaging with a 0.1-second scan in which the tube current is 70 mAs. In imaging a site in which X-rays largely attenuate, such as an abdominal part, a resulting image is poor in quality due to much noise caused by fluctuations in X-rays. For this reason, electron beam scan based X-ray CT devices are treated as high-speed X-ray CT devices exclusively for hearts.

An increase in the number of rows and an expansion of the areas of the detectors in the rotation axis direction in the foregoing MPCT will result in a degraded image quality due to a wider cone angle (an angle by which an X-ray beam expands in the rotation axis direction), thereby leading to a need for a three-dimensional reconstruction algorithm and a significantly increased processing time. In addition, the expansion of the area of a detector is accompanied with problems such as a lower yield rate due to the use of a large quantity of photodiodes, which are parts of the detector, thus leading to a higher cost.

On the other hand, for reducing the element size with the intention of improving the resolution, a separator is required for dividing the element. Then, the use of this separator results in a reduced amount of incident rays, causing a lower use efficiency of irradiated X-rays. Also, noise increases due to an insufficient dose, resulting in a lower quality of tomographic images.

With this being the situation, a quarter offset may be employed to provide images at a higher spatial resolution than when the quarter offset is not used. However, the resolution of projection data depends on the size of device elements, and the resulting resolution is approximately 25% at most. Also, since the quarter offset improves the resolution using opposite data, no effect can be produced for a half reconstruction (reconstruction with projection data for 180° phase) using no opposite data, and the like. In addition, when helical scan imaging is performed, the effect is reduced because opposite positions move to the rotary axis. Similarly, an approach for adjusting a helical pitch has also been proposed for producing similar effects to the quarter offset for the rotation axis resolution, this approach has similar problems to the quarter offset.

### DISCLOSURE OF THE INVENTION

It is an object of the present invention to provide a multi-slice X-ray CT device and method which are capable of capturing high-density and high-resolution projection data at high speeds without reducing the X-ray use efficiency.

It is also an object to improve a temporal resolution of helical scan to achieve a higher image quality without making measurements based on a wide cone angle (an X-ray beam expansion angle in the rotation axis direction). It is a further object to provide a multi-slice X-ray CT device and method which are capable of capturing a four-dimensional tomographic image of a heart with less motion artifact due to pulsation of the heart.

To realize the foregoing objects, the present invention configures an X-ray CT device in the following manner.

(1) A multi-slice X-ray CT device irradiating X-rays while rotating around the outer periphery of a subject about a body axis thereof substantially as a rotation axis and detecting X-rays which transmit through the subject, wherein the multi-slice X-ray CT device comprises:
a plurality of pairs of X-ray sources and detector rows, wherein the X-ray sources are capable of irradiating X-rays, and the detector rows are disposed opposite to the X-ray sources across the subject and have a single row or multiple rows of detectors for detecting X-rays irradiated from the X-ray sources and transmitting through the subject to generate signals representative of the detected X-rays;
a bed for carrying the subject, movable in the rotation axis direction relatively to the plurality of pairs of X-ray sources and detector rows; and
an image reconstruction unit for processing the signals to create an image, the multi-slice X-ray CT device wherein,
at least one of the plurality of detector rows is a multi-row detector, and the plurality of detector rows are the same as or different from one another in the width in a rotating direction, the number of rows, and the width of the detector rows.

(2) The multi-slice X-ray CT device described in (1), wherein a mutual positional relationship among the plurality of pairs of X-ray sources and detector rows is controlled in the rotation axis direction in accordance with a desired region of interest.

(3) The multi-slice X-ray CT device described in (1) or (2), wherein at least the X-ray sources or the detector rows are moved relative to the subject to control the mutual positional relationship among the plurality of pairs of X-ray sources and detector rows.

(4) The multi-slice X-ray CT device described in any of (1) to (3), wherein the plurality of pairs of X-ray sources and detector rows are three pairs, a rotation phase difference between the respective pairs is 120°, and the plurality of pairs can be simultaneously rotated while the rotation phase difference is maintained.

(5) The multi-slice X-ray CT device described in claim 3, wherein at least two of the number of slices in the rotation axis direction, an offset coefficient which represents a degree to which at least the X-ray sources or the detector rows are moved relatively to the subject, and a helical pitch can be set from the outside.

(6) The multi-slice X-ray CT device described in any of (2) to (5), wherein the multi-slice X-ray CT device can be set in a high speed imaging mode, a rotation axis direction resolution preference mode, or a temporal resolution preference mode.

(7) The multi-slice X-ray CT device described in any of (1) to (6), wherein the image reconstruction unit substitutes real data for projection data at opposite positions on the rotation phase in the signal processing.

(8) The multi-slice X-ray CT device described in any of (1) to (6), wherein the image reconstruction unit performs the reconstruction by combining data at different rotation phases in the same slice upon weighted helical correction reconstruction in the signal processing.

(9) The multi-slice X-ray CT device described in any of (1) to (4), wherein:
for conducting high speed imaging in reconstructing an image of the region of interest, the offset coefficient, which is a degree to which at least the X-ray sources or the detector rows are moved relatively to the subject, is set to a large integer so as to expand a range to be dynamically imaged within the region of interest and simultaneously narrow down a region within the region of interest in which a high temporal resolution is desired,
the offset coefficient is set to a value less than one when a resolution in the rotation axis direction is increased in order to narrow down the range to be dynamically imaged and simultaneously increase a number into which a slice is divided on data processing, and
the offset coefficient is set to a small integer when a high temporal resolution is desired widely in the rotation axis direction so as to narrow down the range to be dynamically imaged within the region of interest and simultaneously expand the range within the region of interest in which a high temporal resolution is desired.

(10) The multi-slice X-ray CT device described in any of (1) to (6), wherein the scan cycle and the number of rows in the detector rows are determined from measured heart rate data of the subject, divided projection data substantially equal in heart phase are collected based on the scan cycle and the number of rows in the detector rows, and a tomographic image of the heart at an arbitrary slice position is created based on the divided projection data in the image reconstruction unit.

Other objects, features, and advantages of the present invention will become apparent from the following description of embodiments of the present invention taken in conjunction with the accompanying drawings.

### BRIEF DESCRIPTION OF THE DRAWINGS

FIGS. 1A, 1B are diagrams illustrating the configuration of X-ray tubes and multi-slice detectors according to one embodiment of the present invention;

FIGS. 2A, 2B, 2C are diagrams showing the relationship among the X-ray tubes, multi-slice detectors illustrated in FIGS. 1A, 1B, and slice collimators;

FIG. 3 is a block diagram illustrating a system block of the X-ray tubes and multi-slice detectors illustrated in FIGS. 1A, 1B;

FIGS. 4A, 4B are an explanatory diagram of a high voltage generator for the X-ray tube, and a block diagram illustrating an imaging procedure according to one embodiment of the present invention. In FIG. 4A, 31 designates a multi-slice detector A; 32 a multi-slice detector B; and 33 a multi-slice detector C;

FIGS. 5A, 5B are diagrams illustrating a measurement system for the X-ray tube and multi-slice detector illustrated in FIGS. 1A, 1B;

FIGS. 6A-6E are diagrams showing a dynamic scan by the X-ray tubes and multi-slice detectors illustrated in FIGS. 1A, 1B. FIG. 6A shows when an offset coefficient N is zero (N=0), and a dynamic range spans eight slices; FIG. 6B when the offset coefficient N is one (N=1), and the dynamic range spans six slices; FIG. 6C when the offset coefficient N is two (N=2), and the dynamic range spans four slices; FIG. 6D when the offset coefficient N is three (N=3), and the dynamic range spans two slices; and FIG. 6E shows when the offset coefficient N is 0.33 (N=0.33), and dynamic range spans 24 slices;

FIGS. 7A, 7B are diagrams showing an example of high speed imaging by the multi-slice X-ray CT device illustrated in FIGS. 2A-2C. 31A, 31B, 31C in FIG. 7B designate multi-slice detectors;

FIGS. 8A, 8B are diagrams showing another example of high speed imaging by the multi-slice X-ray CT device illustrated in FIGS. 2A-2C;

FIGS. 9A, 9B are diagrams showing the relationship between a fan beam and a parallel beam by the X-ray tube and multi-slice detector illustrated in FIGS. 1A, 1B;

FIGS. 10A, 10B are diagrams showing the relationship between a fan beam and a parallel beam by the X-ray tube and multi-slice detector illustrated in FIGS. 1A, 1B;

FIGS. 11A, 11B are diagrams showing another example of high speed imaging by the multi-slice X-ray CT device illustrated in FIGS. 2A-2C;

FIGS. 12A, 12B are diagrams showing another example of high speed imaging by the multi-slice X-ray CT device illustrated in FIGS. 2A-2C;

FIGS. 13A, 13B are diagrams showing an example of high density imaging by the multi-slice X-ray CT device illustrated in FIGS. 2A-2C;

FIGS. 14A, 14B are diagrams showing another example of high density imaging by the multi-slice X-ray CT device illustrated in FIGS. 2A-2C;

FIGS. 15A, 15B are diagrams showing another example of high density imaging by the multi-slice X-ray CT device illustrated in FIGS. 2A-2C;

FIGS. 16A, 16B are diagrams illustrating an example of high temporal resolution imaging by the multi-slice X-ray CT device illustrated in FIGS. 2A-2C;

FIGS. 17A, 17B are diagrams illustrating another example of high temporal resolution imaging by the multi-slice X-ray CT device illustrated in FIGS. 2A-2C;

FIGS. 18A, 18B are diagrams showing an exemplary imaging operation by three pairs of X-ray tubes and multi-slice detectors;

FIG. 19 is a diagram illustrating a layout when there are six pairs of the X-ray tubes and multi-slice detectors illustrated in FIGS. 1A, 1B;

FIG. 20 is a diagram illustrating a processing flow for capturing a high resolution image by the multi-slice X-ray CT device illustrated in FIGS. 1A, 1B;

FIG. 21 is a diagram showing a method of generating high resolution projection data by the multi-slice X-ray CT device illustrated in FIGS. 1A, 1B;

FIG. 22 is a diagram illustrating a method of generating high resolution projection data by the multi-slice X-ray CT device illustrated in FIGS. 1A, 1B;

FIGS. 23A-23D are diagrams illustrating a method of generating high resolution projection data by the multi-slice X-ray CT device illustrated in FIGS. 1A, 1B;

FIG. 24 is a diagram illustrating the configuration of one embodiment of the multi-slice X-ray CT device illustrated in FIGS. 1A, 1B;

FIGS. 25A-25C are diagrams for explaining a method of reconstructing an image from projection data of the multi-slice X-ray CT device illustrated in FIGS. 1A, 1B;

FIG. 26 is a diagram illustrating a processing flow in another embodiment;

FIGS. 27A, 27B are diagrams showing a circular trajectory scan and a helical trajectory scan;

FIGS. 28A, 28B are diagrams showing measurement trajectory diagrams when a spiral trajectory is interpolated to a circular trajectory for reconstruction in accordance with one embodiment of the present invention, where FIG. 28A shows a measurement trajectory when three tubes and one row are used with a spiral pitch P=6; and FIG. 28B shows a measurement trajectory when three tubes and three rows are used with the spiral pitch P=18;

FIGS. 29A-29H are diagrams showing weights for a helical correction of the measurement trajectories shown in FIGS. 28A, 28B, where FIGS. 29D, 29H show the result of combination when three tube balls and three rows are used with the pitch P=18;

FIGS. 30A, 30B are diagrams illustrating the shapes of the helical correction weights shown in FIGS. 29A-29D;

FIGS. 31A, 31B are diagrams showing unit data in a uniform angle positioning, where FIG. 31A shows the unit data when there is one pair of the X-ray tube and multi-slice detector, and FIG. 31B shows the unit data when there are three pairs;

FIG. 32 is a diagram showing a trajectory of projection data by a multi-tube multi-slice X-ray CT device which has multi-slice detectors disposed at angular intervals of 120°;

FIG. 33 is a diagram showing exemplary weighting functions for generating a satisfactory image in a multi-tube multi-slice X-ray CT device which is one embodiment of the present invention, where an area indicated by a broken line shows an example of correcting discontinuity by reducing weighting coefficients;

FIG. 34 is a diagram showing the proportion of weights occupied by projection data of respective tubes in a correction in accordance with one embodiment of the present invention, where an area indicated by a broken line shows an example of correcting discontinuity by reducing weighting coefficients;

FIG. 35 is a diagram illustrating the whole configuration of a conventional X-ray CT device; and

FIGS. 36A, 36B are diagrams illustrating a combination of a conventional X-ray CT device with an ECG gate scan.

### BEST MODE FOR CARRYING OUT THE INVENTION

While the following description is made on the case of three tube balls, it should be understood that the present invention can be applied to another number of pairs based on the following description as long as there are a plurality of pairs of X-ray tubes and detectors, and a plurality of pairs other than three pairs are included in the scope of the right based on the present application.

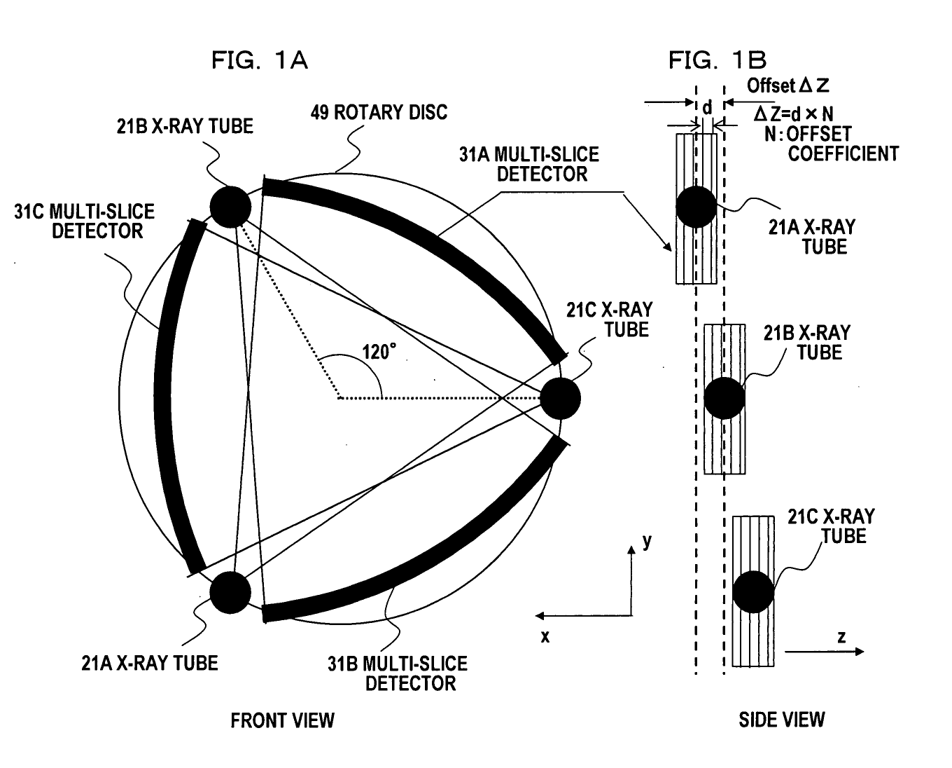

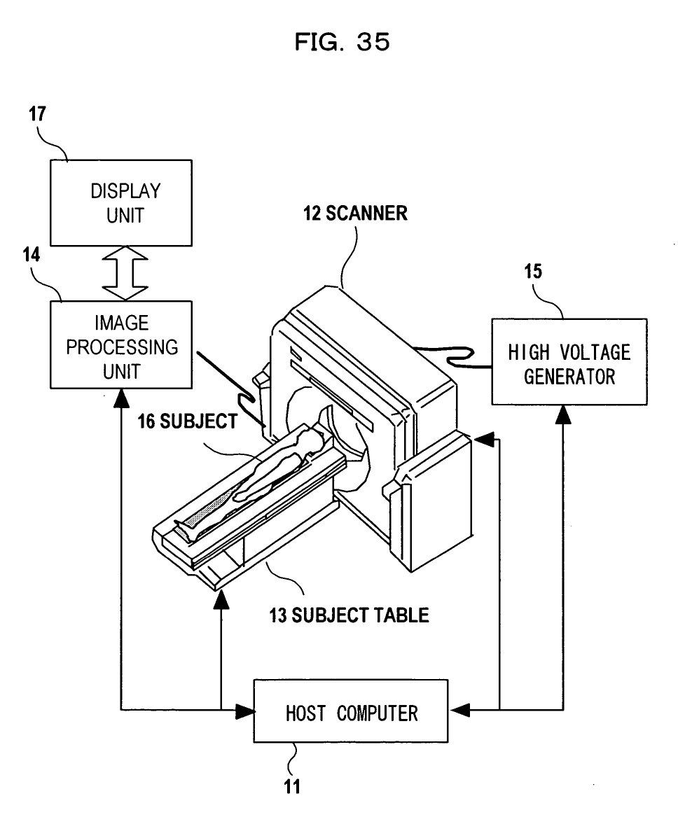

FIGS. 1A, 1B are diagrams illustrating the configuration (three-tube system) of three pairs of X-ray tubes 21A, 21B, 21C and multi-slice detectors (two-dimensional multi-slice detectors) 31A, 31B, 31C of a multi-slice X-ray CT device in this embodiment. Since the X-ray CT of the present invention is identical in the basic configuration to FIG. 35, the same numerals are used for common components. As illustrated in a front view of FIG. 1A, three pairs of X-ray tubes 21A-21C and multi-slice detectors 31A-31C are mounted on a rotary disc 49 installed in a scanner unit 12 with a rotation phase difference of 120°.

Then, the three pairs or sets are simultaneously rotated while maintaining their relative positional relationship of imaging geometry such as the distance between the X-ray tubes 21A-21C and multi-slice detectors 31A-31C, the distance between the X-ray tubes 21A-21C and the center of rotation, and the like.

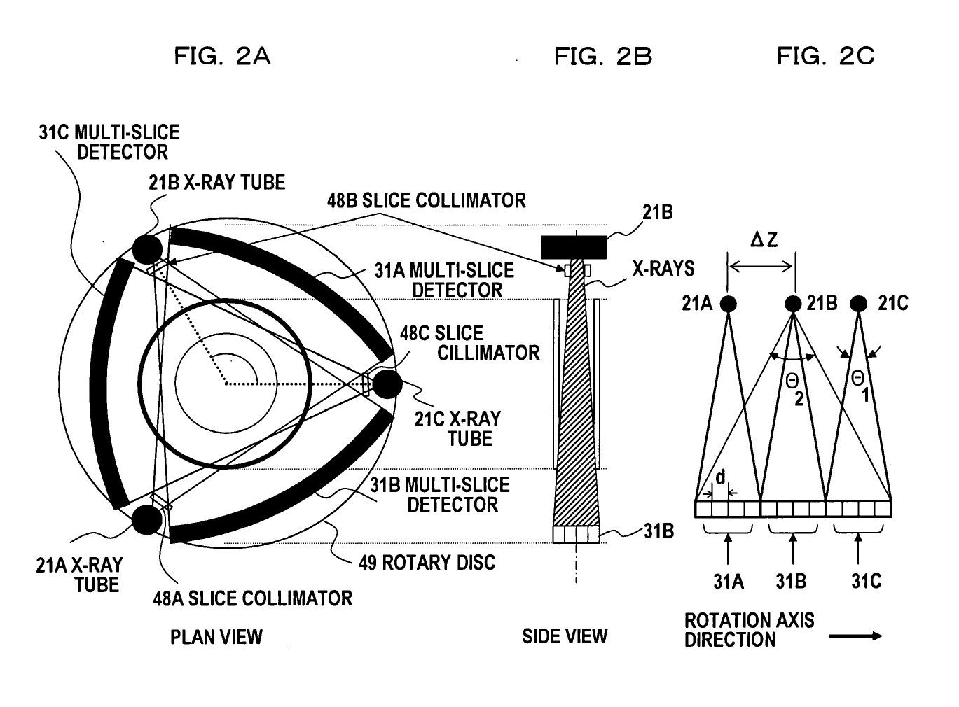

Also, X-rays are irradiated from the X-ray tube 21A to a subject 16 being recumbent on a subject table 13. The X-rays are provided with directivity by a slice collimator 48A (FIG. 2A) and detected by the multi-slice detector 31A, and in this event, data of X-ray transmitting the subject 16 is detected using the multi-slice detector 31A while changing the angle at which the X-rays are irradiated by rotating the disc 49 about the subject 16.

Then, a tube current which can be applied to one X-ray tube 21A is determined by the size of a target (focus size) which is the source of the X-rays, the rotational speed of a rotary anode, and the like. Therefore, as the diameter of the target is increased, the rotational speed is increased with more difficulties in respect to the life time, deflected rotations, and the like of bearings, so that a maximum tube current is limited.

However, in the three-tube multi-slice CT device of this embodiment, since X-rays from the three X-ray tubes 21A-21C do not interfere with one another, the X-rays can be simultaneously emitted. It is therefore possible to mount a small X-ray tube 21A of approximately 2 MHU (mega heat unit), by way of example, and apply each of the three X-ray tubes 21A-21C, for example, with a tube current of 350 mA to readily provide an irradiation dose with the tube current of 1000 mA or more.

Also, as illustrated in a side view of FIG. 1B, one feature of this embodiment lies in that the three pairs of X-ray tubes 21A-21C and multi-slice detectors 31A-31C can be disposed with an offset in the rotation axis Z-direction. Then, a deviation (offset) ΔZ in the rotation axis Z-direction can be expressed by ΔZ=d×N, where d is the thickness of one of rows (slices) of the multi-slice detectors 31A-31C, and N is an offset coefficient.

With this configuration of FIGS. 1A, 1B, by offsetting each of three pairs or sets of X-ray tubes 21A-21C and multi-slice detectors 31A-31C in the rotation axis direction, and rotating them relative to the subject on the subject table, a three-dimensional tomographic image can be created for a region of interest of the subject 16.

FIGS. 2A-2C in turn are diagrams illustrating the configuration of the X-ray tubes 21A-21C, multi-slice detectors 31A-31C, and slice collimators 48A-48C. As illustrated in a plan view of FIG. 2A, the slice collimators 48A-48C are associated with the X-ray tubes 21A-21C, respectively. Then, as illustrated in a side view of FIG. 2B, a relationship such as the X-ray tube 21B and multi-slice detector 31B, for example, can be established in a cross-section in the rotation axis Z-direction. Specifically, X-rays emitted from the X-ray tube 21B are limited in a slice direction (rotation axis Z-direction) by the slice collimator 48B, and impinges on the multi-slice detector 31B which opposes the X-ray tube 21B. Then, the multi-slice detector 31B measures projection data of a plurality of cross-sections (multiple slices).

Then, if the width of the detector (number of rows L) matches the deviation ΔZ in the rotation axis direction during a helical scan, a range of 3×L rows (in the figure, L=4, and 12 rows) can be simultaneously measured, as illustrated in FIG. 2C. Further, each projection data in this event has a small expansion angle (cone angle) θ₁ of an X-ray beam in the rotation axis Z-direction, and projection data equivalent to a large cone angle θ₂ can be formed only with the projection data of the small cone angle θ₁. This improves a temporal resolution of the helical scan, reduces a cone angle distortion, and realizes a higher image quality.

Further, three pairs or sets of X-ray tubes 21A-21C and multi-slice detectors 31A-31C are translated relative to the rotation axis, or are maintained at rest. Then, a multi-tube three-dimensional tomographic imaging device is realized, where conical or pyramidal X-rays, which are three-dimensionally divergent, are irradiated (fan beam) from the three pairs of X-ray tubes 21A-31C to the subject 16, a radiation irradiation field in the rotation axis direction is limited in accordance with a region of interest of the subject 16 using the slice collimators 48A-48C, the X-rays which have transmitted the subject 16 are detected using the two-dimensionally arranged multi-slice detectors 31A-31C, and a three-dimensional tomographic image is created from projection data detected by the multi-slice detectors 31A-31C.

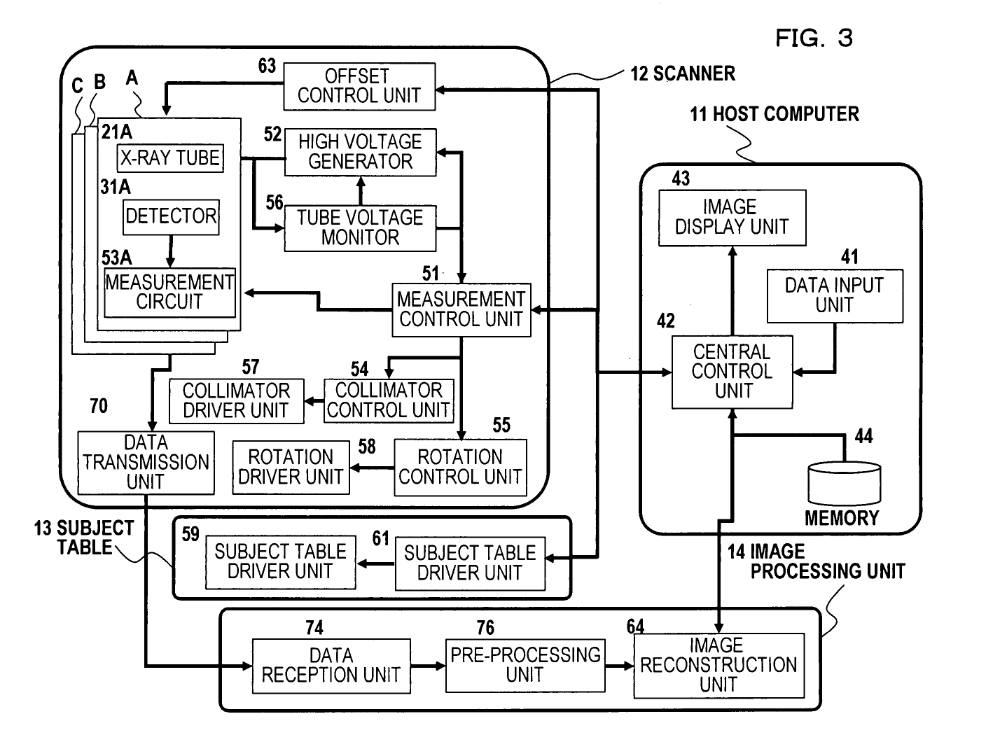

FIG. 3 is a block diagram illustrating a system block of this embodiment. As illustrated in FIG. 3, the multi-slice X-ray CT device system comprises a host computer 11, a scanner 12, a subject table 13, and an image processing unit 14.

Then, in accordance with imaging conditions selected by the operator through a data input unit 41 included in the host computer 11, the central control unit 42 issues instructions to a measurement control unit 51, a subject table control unit 61, and an image reconstruction unit 64. The measurement control unit 51 indicates X-ray conditions sent from the central control unit 42 to the high voltage generator 52, indicates a timing of X-ray emission from the X-ray tube 21A and the start of measurement to a measurement circuit 53A, and provides indications to a collimator control unit 54 and a rotation control unit 55.

Also, as illustrated in FIG. 3, the X-ray tubes 21A-21C, multi-slice detectors 31A-31C, and measurement circuits 53A-53C are configured in three pairs, and outputs of the measurement circuits 53A-53C are transmitted to a data transmission unit 70. Then, transmission data from the data transmission unit 70 is transmitted to a data reception unit 74, and a tomographic image of the subject 16 is created by a pre-processing unit 76 and the image reconstruction unit 64. Then, the resulting tomographic image is processed by the central control unit 42, and displayed on an image display unit 43 for use in diagnosis. The result of the processing is also stored in a memory 44.

On the other hand, a tube voltage of the X-ray tube 21A is measured by a tube voltage monitor 56, and the result of the measurement is fed back to the high voltage generator 52 to control the X-ray dose by the X-ray tube 21A. Also, each driver unit is controlled by each control unit, i.e., a collimator driver unit 57 is controlled by the collimator control unit 54; a rotation driver unit 58 by a rotation control unit 55; and the subject table driver unit 59 by the subject table control unit 61, respectively. The offset control unit 63 controls an offset in the rotational axis direction of the X-ray tubes and X-ray detectors.

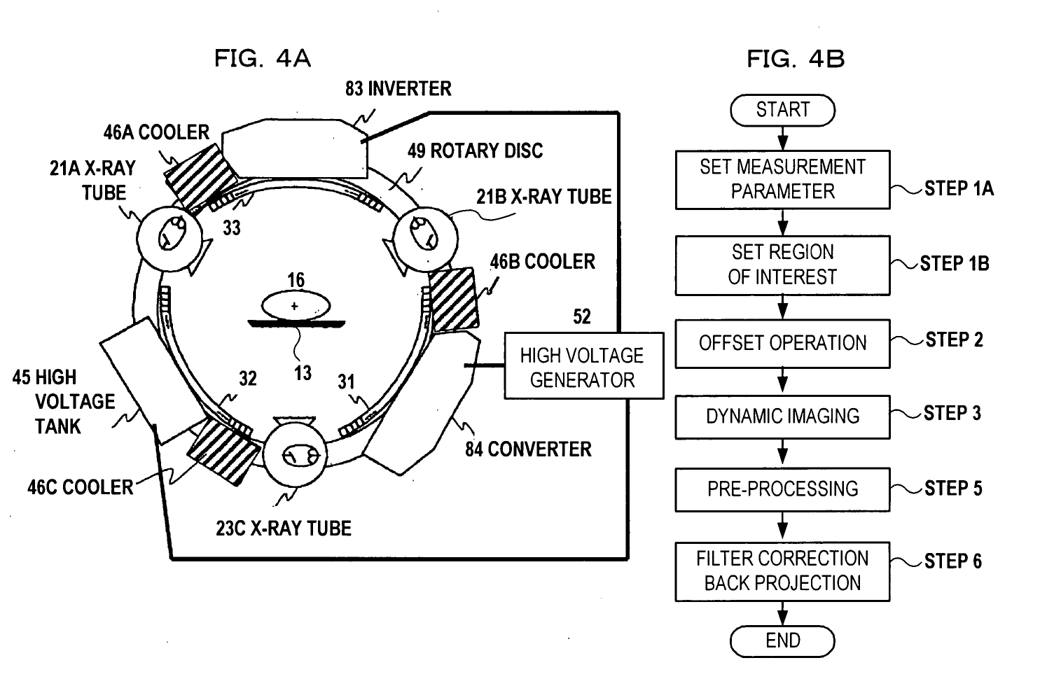

FIG. 4A is a diagram illustrating the system configuration including the high voltage generator 52 of this embodiment. As illustrated in FIG. 4A, the subject 16 on the subject table 13 is moved in the rotation axis direction. Then, each of the X-ray tubes 21A-21C is supplied with electric power from the same high voltage generator 52. Also, as shown by the present applicant (see Japanese Patent Application No. 2001-280489), an inverter unit 83, a converter unit 84, and coolers 46A-46C are separated to realize an optimal weight balance, making it possible to reduce burdens on the rotary disc 49.

As another means, a high voltage tank 45 alone may be installed, while the inverter unit 83 may be disposed in a static system, in order to reduce the weight of the body of rotation. A saving in space can be achieved by integrating or reducing in size the measurement circuits 53A-53C (FIG. 3) connected to the multi-slice detectors 31A-31C.

The three multi-slice detectors 31A-31C simultaneously measure projection data of the subject 16. Since the number of views is preferred to be a multiple of three, this embodiment employs 900 views per rotation. The data rate per multi-slice detector is calculated to be 1500 views/second for 0.6 seconds of rotation. Since three data sets are simultaneously measured, the data transfer rate to the static system is calculated to be 4500 views/second. Assuming 1024 channels, 16 slices, and 16 bits/data, the data transfer rate is approximately 1.1 Gbps.

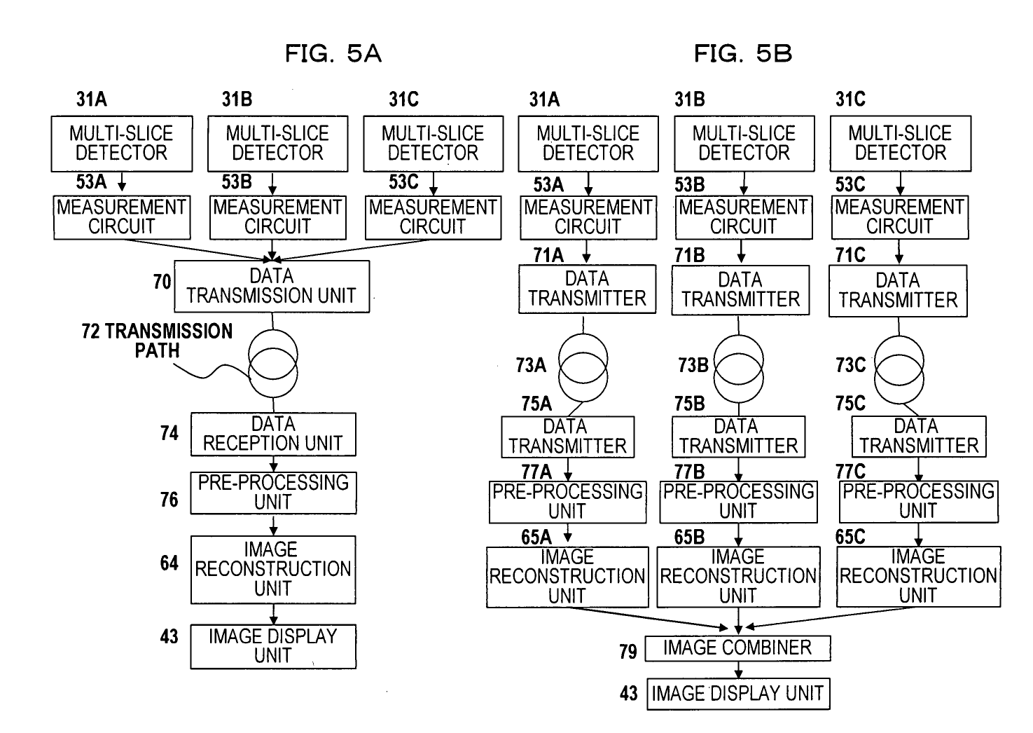

Next, FIGS. 5A, 5B are diagrams illustrating a measurement system of the X-ray tube and multi-slice detector in this embodiment. As illustrated in FIG. 5A, three sets of data measured by three multi-slice detectors 31A-31C and measurement circuits 53A-53C are bundled in the data transmission unit 70, and is transferred through a transmission path as single serial data.

The data reception unit 74 separates each projection data corresponding to the three pairs from the serial data for transfer to the pre-processing unit 76. The pre-processing unit 76 performs offsetting, air calibration, log conversion, and the like. The air calibration should be performed for each combination of the X-ray tubes 21A-21C and multi-slice detectors 31A-31C. The image reconstruction unit 64 calculates a tomographic image of a desired slice using a known multi-slice helical reconstruction algorithm. Then, the tomographic image of the subject 16 is displayed on the image display unit 43 for use in diagnosis.

Of course, as illustrated in FIG. 5B, the data transmissions of the three multi-slice detectors 31A-31C and measurement circuits 53A-53C may be received separately by the data receivers 75A-75C by providing three independent data transmitters 71A-71C and transmission paths 73A-734C. In this case, the image reconstruction processing for a number of individually required views may be performed by the separate pre-processing units 77A-77C and image reconstruction units 65A-65C, and then, the resulting images may be added by an image combiner 79. The created image is displayed on the image display unit 43 for use in diagnosis.

Next, description will be made on an imaging method in the multi-slice X-ray CT device of this embodiment

(1) Dynamic Scan

A dynamic scan is an imaging method which sequentially images the same cross-section for observing dynamics, typically, a flow of a contrast medium or the like, and is required to provide a high temporal resolution. In this embodiment, when three pairs of X-ray tubes 21A-21C and multi-slice detectors 31A-31C are mounted on the rotary disc 49, they are positioned with an offset in the rotation axis direction. This deviation (offset) AZ is set by the product of the thickness d of the row (slice) of the multi-slice detectors 31A-31C and the offset coefficient N, as shown in FIGS. 1A, 1B.

Now, a general procedure of data processing according to this application will be described with reference to FIG. 4B. At step 1A, parameters are set for measurements. Here, the parameters include the aforementioned deviation ΔZ, the thickness d of the slice of the detector, and the offset coefficient N. At step 1B, a region of interest is set. In the following, the region of interest refers to a range which is to be dynamically imaged or a range for which a high resolution is desirably achieved.

At step 2, at least one of the X-ray tubes and detectors, which is suited to the offset coefficients set at step 1A, is moved in the rotation axis direction. Here, a mechanism for moving the X-ray tubes can be implemented, for example, by JP-A-09-201352. A signal from the offset control unit in FIG. 3 of this application is directly inputted to a control device 16 in FIG. 1 of JP-A-09-201352 to move the X-ray tubes in the rotation axis direction, thus realizing the offset. Also, as a feature for moving the detectors, a driving mechanism 18 in FIG. 1 of the aforementioned JP-A-09-201352 may be combined with a translating device such as the control device 16 and its driving means such as a motor on the rotary disc of a gantry, and a detector is mounted on the translating device to make it drivable. Further, as a feature for moving the detectors and X-ray tubes in pair, a number of members for simultaneously holding the detector and X-ray tube (rotary disc and gantry) may be provided as much as the number of pairs, as shown in FIG. 10 of JP-A-06-038957, and driving means may be provided such that they can be individually moved. Alternatively, as mentioned above, a plurality of detectors and a plurality of X-ray tubes are all made individually movable in the rotation axis direction on a single gantry, and may be separately controlled by the central control unit 42 or offset control unit 63 such that a pair of corresponding detector and X-ray tube moves by the same distance in the rotating direction. Assume that the movement can be finely set smaller than the width of the row of the foregoing movement minimal pitch detector.

At step 3, dynamic imaging is performed, and at step 5 pre-processing is performed for forming an image. At step 6, filter correction back projection is performed for reconstructing an image.

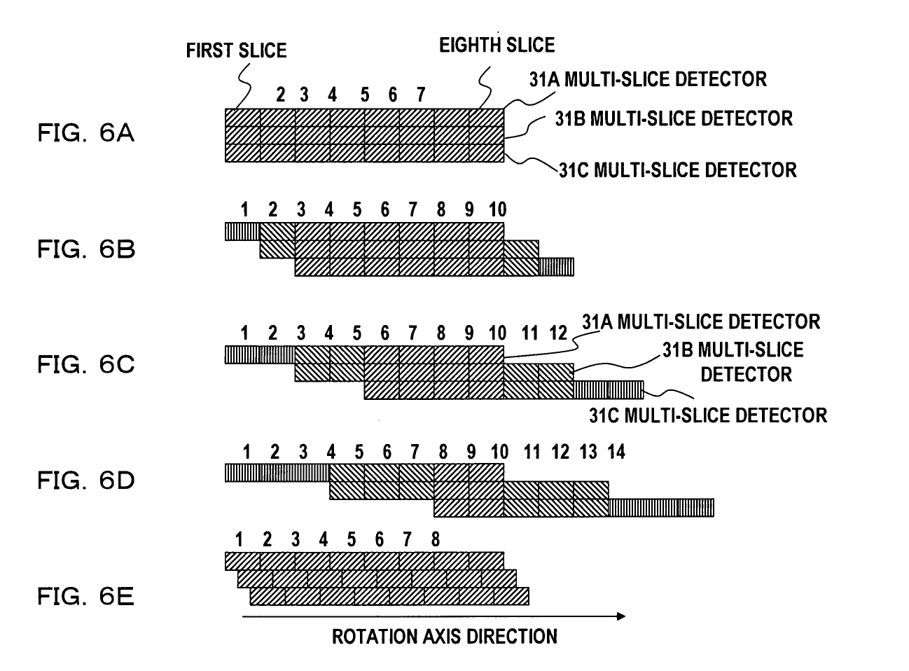

FIGS. 6A-6D are diagrams showing examples of the offset coefficient N from zero to three (N=0-3). As shown in FIG. 6A, when the offset coefficient N is zero (N=0) (small integer), the eight slices of slices 1-8 of the multi-slice detectors 31A-31C can be fully imaged at a high temporal resolution. However, an imaging range is the same as a range which is imaged when there is one multi-slice detector 31.

Next, when the offset coefficient N is one (N=1) (FIG. 6B), central slices 3-8 are measured by three multi-slice detectors 31A-31C, and images can be captured for six slices at a high temporal resolution. In addition, wide range imaging (over ten slices) is possible for two slices in the rotation axis direction. Also, as compared with the offset coefficient N=0, when the offset coefficient N is one (N=1), the rows of the slices 1 and 10 at both ends are measured by one multi-slice detector 31A, and the rows of the slices 1 and 10 inside of them are measured by two multi-slice detectors 31A and 31B, so that the temporal resolution is degraded as compared with the six central slices. However, the imaging range is expanded over slices 1-10.

Step 5 in FIG. 4B will be described with reference to the case of FIG. 6B. For the second row in FIG. 6B, only the detector rows 31A and 31B are available, when three tubes are used, and data for 360 degrees are available only after they have rotated by 240 degrees. Views which overlap in this event may undergo averaging or the like.

Similarly, as shown in FIG. 6C, only four slices are available at a high temporal resolution when the offset coefficient N is two (N=2), and only two slices are available when the offset coefficient N is three (N=3) (large integer), but their respective imaging ranges span over 12 slices and 14 slices, i.e., a wide range can be imaged. Therefore, the deviation (offset) ΔZ should be selected in accordance with a range which is to be dynamically imaged, or a range in which a high temporal resolution is desired.

In an example of FIG. 6E, three pairs of X-ray tubes 21A-21C and multi-slice detectors 31A-31C are offset by one third of one slice, resulting in N=0.33 (less than one), wherein a dynamic scan can be made with an improved resolution in the rotation axis direction.

(2) Helical Scan

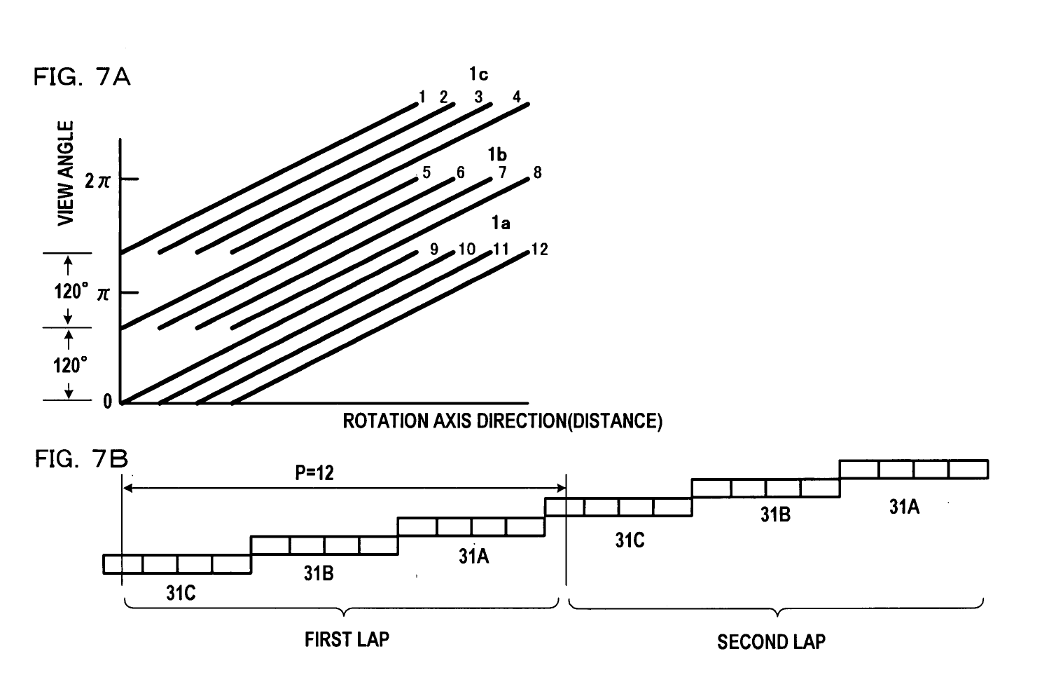

FIGS. 7A, 7B to 12A, 12B, FIGS. 13A, 13B to 15A, 15B, and FIGS. 16A, 16B to 18A, 18B are diagrams showing features of a helical scan in the multi-slice X-ray CT device of this embodiment.

FIGS. 7A, 7B are diagrams showing an example which is suitable for high speed imaging of the multi-slice X-ray CT device of this embodiment. FIG. 7A shows measured trajectories of helical scans of the multi-slice detectors 31A-31C when the vertical axis represents the angle of view (sampling in the rotating direction), and the horizontal axis represents the distance in the rotation axis direction. FIG. 7B in turn shows the positional relationship among the multi-slice detectors 31A-31C when viewed on a line of (4/3)π=240°, wherein the number M of slices in the rotation axis direction (the number of faults matching the number of rows of the multi-slice detectors 31A-31C) is four (M=4) in this embodiment.

In the following, the figure below the diagram of measured trajectories shows a positional relationship among the X-ray detectors when viewed on the line of (4/3)π=240°.

First, a measured trajectory 1a of the multi-slice detector 31A starts with the view at the rotating angle of 0°, has four lines equal to the number of slices (number of faults). Measured trajectories 1b, 1c of the other multi-slice detectors 31B, 31C start from the rotating angles of 120° and 240°, respectively.

In the conditions shown in FIGS. 7A, 7B, a helical pitch (the number of faults over one round of the measured trajectory) P is calculated by the following equation:
P=3×(N+M) (1)
where M: the number of slices in the rotation axis direction of
the multi-slice detector; and
N: the offset coefficient of the deviation (Offset) ΔZ

Then, the pitch P in the rotation axis direction is 12 (P=12), as shown in FIGS. 7A, 7B, and matches the calculated value of Equation (1) (N=0, M=4, P=12). Therefore, since the pitch available with one each of X-ray tube 21 and multi-slice detector 31A having four arrays (rows) is four (P=4), the pitch P=12 provided in FIGS. 7A, 7B of this embodiment is a number of pitches three times more. Therefore, assuming that a moving speed of the subject table 13 is a speed ratio compared with a one-tube four-row MDCT, the subject table 13 makes measurements possible at a moving speed three times higher in the rotation axis direction. As a result, in this embodiment, a high speed multi-slice X-ray CT device is realized.

When an attempt is made to achieve the pitch P=12, similar to FIGS. 7A, 7B, with one each of conventional X-ray tube 21A and multi-slice detector 31A having four arrays (rows), a multi-slice detector 31A having 12 rows is required. Increasing the number of rows by a factor of three is equivalent to making measurements at a cone angle three times larger, as has been described in FIG. 2C. In other words, in the embodiment shown in FIG. 7A, 7B, measurements can be made at a narrow cone angle, thus realizing a high speed multi-slice X-ray CT device without degrading the spatial resolution in the rotation axis direction.

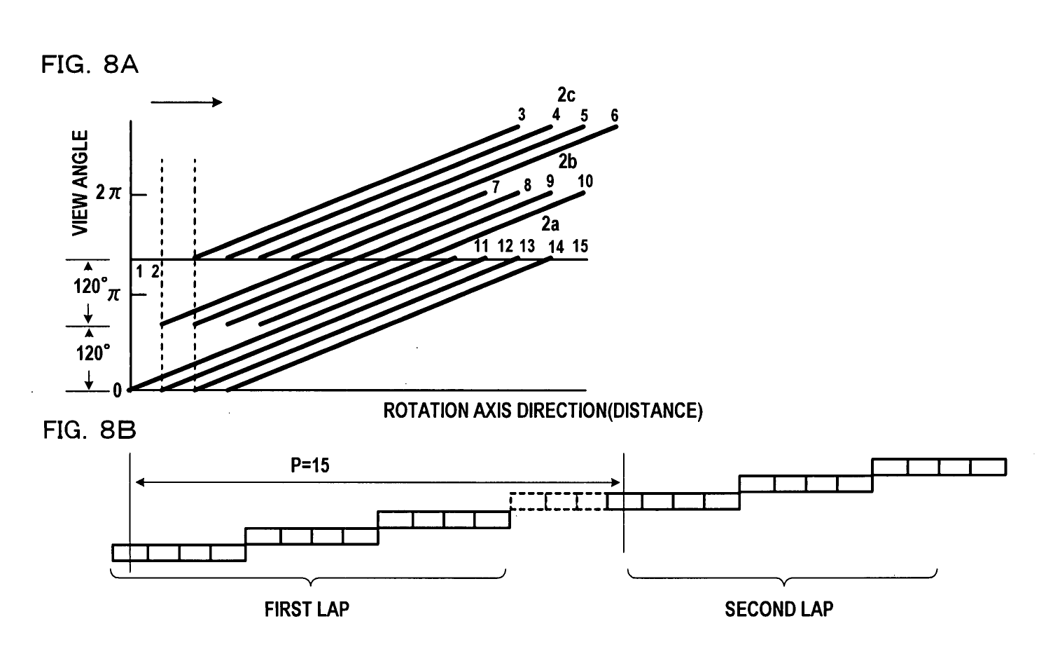

Next, FIGS. 8A, 8B are diagrams showing another example suitable for high speed imaging of the multi-slice X-ray CT device of this embodiment. FIGS. 8A, 8B show the case where the offset coefficient N of the deviation (Offset) ΔZ is one (N=1), and the number M of slices in the rotation axis direction of the multi-slice detector 31A is four (M=4) in Equation (1).

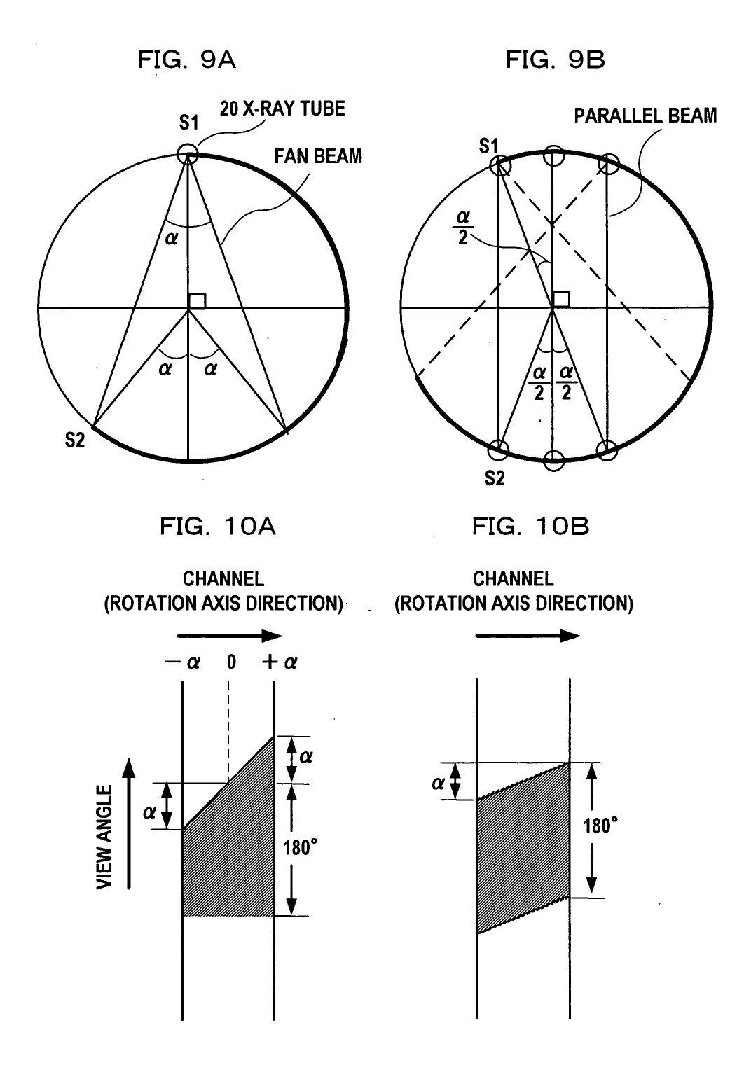

Here, a fan beam and a parallel beam will be described. In FIGS. 9A, 9B, 10A, 10B, α indicates a fan angle. FIGS. 9A, 9B are diagrams showing the relationship between the fan beam and parallel beam. As shown in FIG. 9A, in the multi-slice X-ray CT device, X-rays are irradiated from a target (infinitesimal focal point) of an X-ray tube 20 in a conical or a pyramidal shape, so that an X-ray beam can be regarded as a fan-shaped (sector) beam as shown in FIG. 9A, as observed from the rotation axis direction of the X-ray tube 20. The X-ray beam in a fan shape as viewed from the rotation axis direction is imaged over 360° while it is rotated. In this event, when X-ray beams (S1-S2) irradiated in the same vector direction, viewed from the rotation axis direction, are collected, a parallel beam can be virtually created as shown in FIG. 9B. This processing is generally referred to as “rebinning.”

While the reconstruction of an image generally involves projection data for 360° phase, there is an approach for reconstructing an image with projection data for 180° phase making use of the redundancy with opposite projection data (opposite data). This is referred to as “half reconstruction.” With parallel beams, from the fact that projection data at each phase matches parallel beams centered at the rotation axis and positioned at opposite phases, all projection data of parallel beams just for 180° phase can be reconstructed as projection data for one cycle. On the other hand, with a fan beam, as shown in FIG. 9A, X-ray beams are required over a phase from S1 to S2 (180°+fan angle α), and this phase data (group of fan beam projection data) includes redundant X-ray beam data viewed from the rotation axis direction.

Therefore, X-ray beams must be selected such that the redundancy is constant in the group of fan beam projection data, or normalization must be performed through weighting or the like.

Accordingly, FIGS. 10A, 10B show data ranges of a fan beam and a parallel beam minimally required for reconstructing an image. A bold line in FIG. 10A indicates parallel beam data. Upper and lower bold lines in FIGS. 10A, 10B are in a complementary relationship. In sinogram (diagram which represents projection data with the horizontal axis representing a channel direction and the vertical axis representing a phase direction), data is represented at positions as shown in FIGS. 10A, 10B. From this fact, a data range as shown in FIG. 10B is used in a half scan using a parallel beam, while a data range as shown in FIG. 10A is used in a half scan of a fan beam.

With this half scan using a parallel beam, projection data at seventh to tenth rows are used as opposite data in FIGS. 8A, 8B. The pitch P is 15 (P=15) in the rotation axis direction in the first round, as shown in FIGS. 8A, 8B, which matches the calculated value (N=1, M=4, P=15) by Equation (1). Therefore, a high speed multi-slice X-ray CT device is realized in this embodiment, as compared with the pitch P=4 which is achieved by one each of X-ray tube 21A and multi-slice detector 31A, and with the pitch P=12 when the offset coefficient N is zero (N=0).

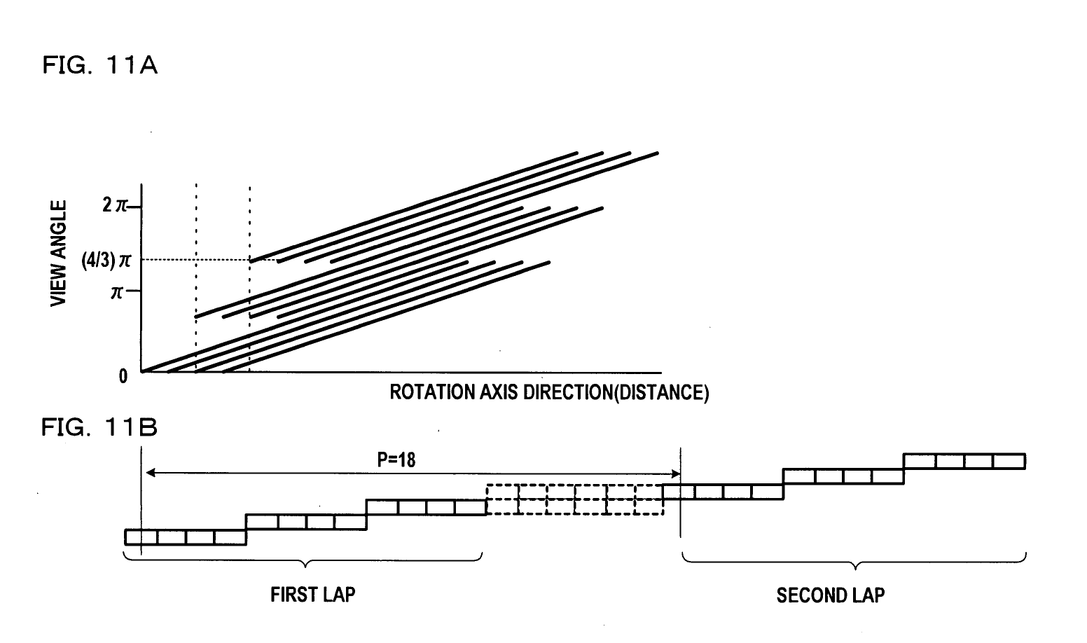

FIGS. 11A, 11B in turn show the case where the offset coefficient N is two (N=2). As shown in FIGS. 11A, 11B, when the offset coefficient N is two (N=2), the pitch P=18 is achieved, thus realizing a high speed multi-slice X-ray CT device.

In the foregoing examples, the offset coefficient N and pitch P are (N=0, P=12) in FIGS. 7A, 7B, (N=1, P=15) in FIGS. 8A, 8B, and (N=2, P=18) in FIGS. 11A, 11B, any of which shows that the resulting performance exceeds the maximum pitch P=8 which is achieved by one each of detector 30 and X-ray tube 20.

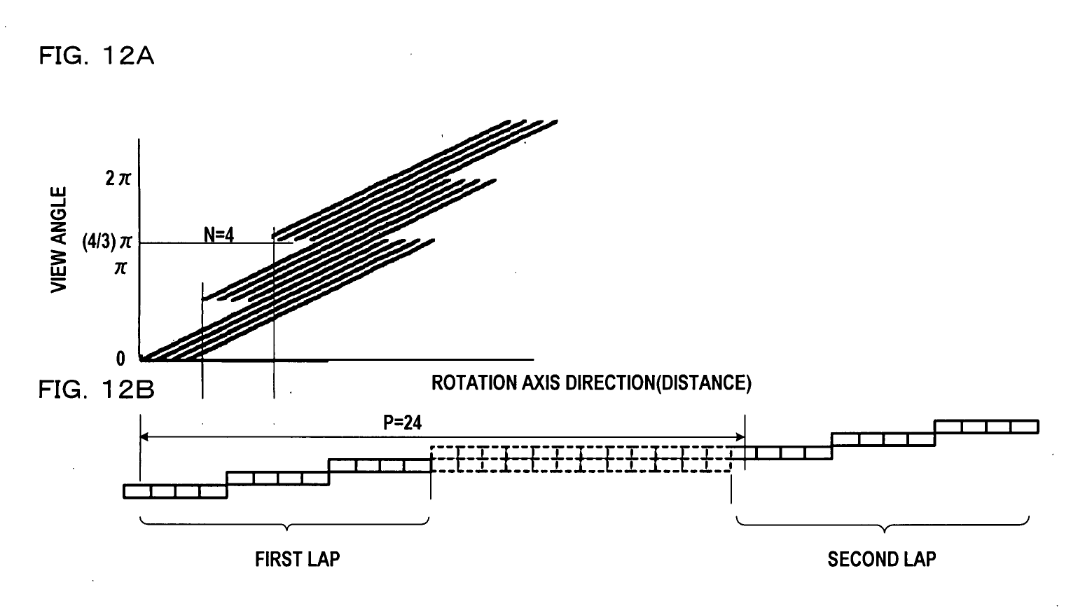

Now, FIGS. 12A, 12B show the case where the offset coefficient N is four (N=4). As shown in FIGS. 12A, 12B, the pitch P=24 can be maximally provided with the offset coefficient N=4. This is the performance effectively equivalent to the number of rows three times as much. This leads to the realization of a reduction in size and price of the multi-slice detectors 31A-31C.

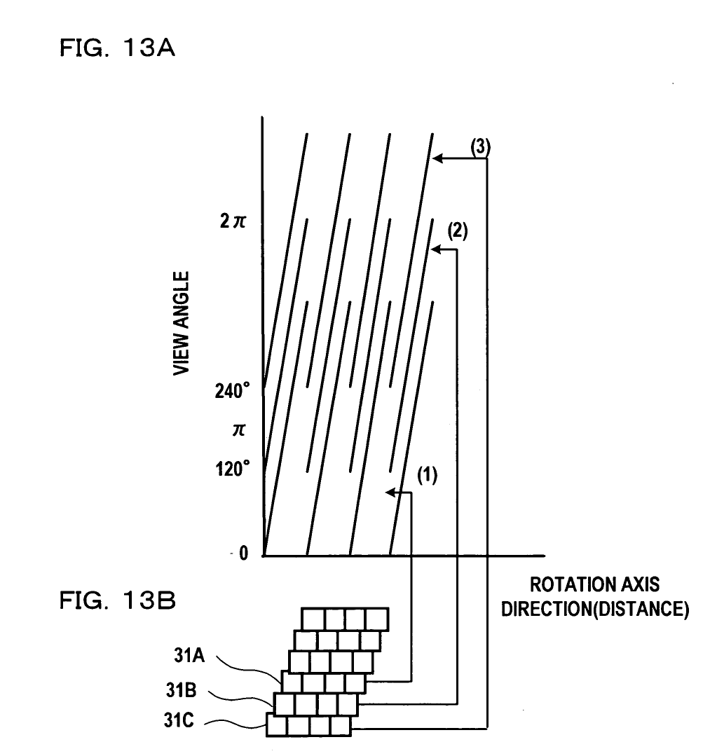

FIGS. 13A, 13B to 15A, 15B are diagrams which show examples suitable for improving the density in the rotation axis direction.

FIGS. 13A, 13B show an example of high density imaging by the multi-slice X-ray CT device. As shown in FIGS. 13A, 13B, three multi-slice detectors 31A-31C are positioned with an offset of N=1/3 rows in the rotation axis direction, resulting in projection trajectories (1)-(3) which represent projection data offset in the rotation axis direction. As a result, the density of data sampling in the rotation axis direction is three times as high as a single multi-slice detector between 240° and 360°, thus enabling high-density and high-quality tomographic imaging.

The helical pitch P is calculated by the following equation:
P=3×N+1 (2)
where N: the offset coefficient of the deviation (offset) ΔZ

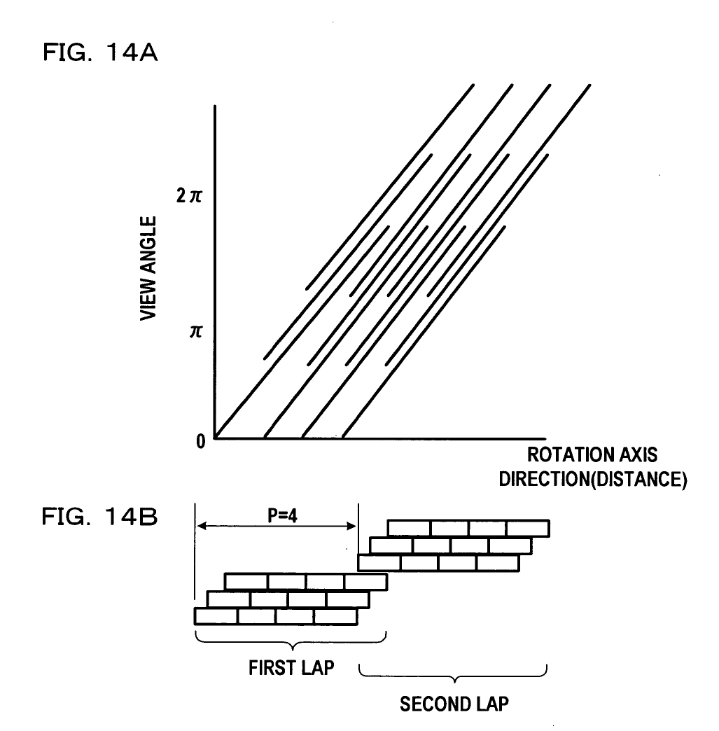

Then, FIGS. 14A, 14B show diagrams when the three multi-slice detectors 31A-31C are disposed with the offset coefficient N=1. As shown in FIGS. 14A, 14B, in this event, the pitch P=4 is derived from Equation (2), thus enabling high-density and high-quality tomographic imaging.

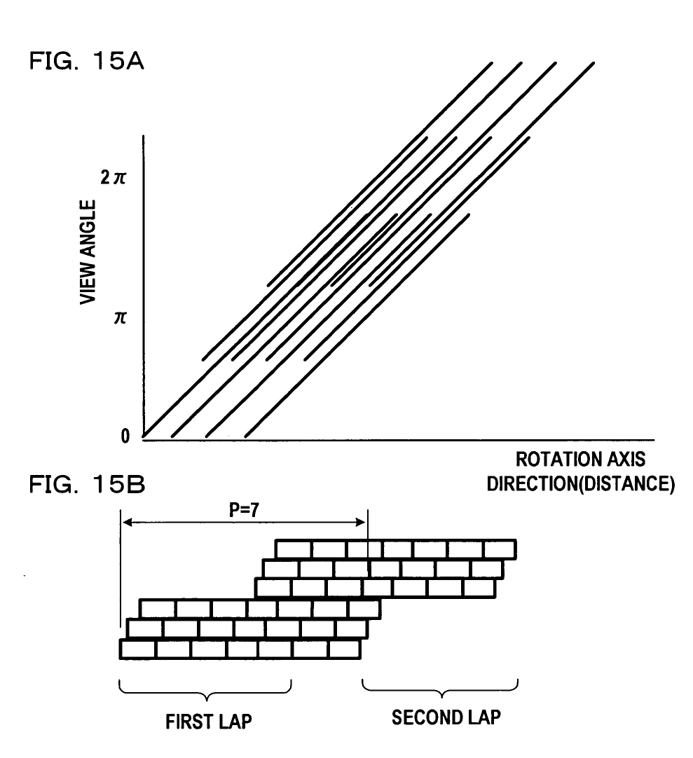

Further, FIGS. 15A, 15B show diagrams when three multi-slice detectors 31A-31C are disposed with the offset coefficient N=2. As shown in FIGS. 15A, 15B, in this event, the pitch P=7 is derived from Equation (2), thus realizing a higher-density and higher-quality multi-slice X-ray CT device.

Assuming herein that the sampling density in the rotation axis direction is the ratio to a one-tube type, the sampling density in the rotation axis direction is three times as high as the one-tube type, in either of the cases shown in FIGS. 13A, 13B to 15A, 15B, thus providing an improvement on the accuracy.

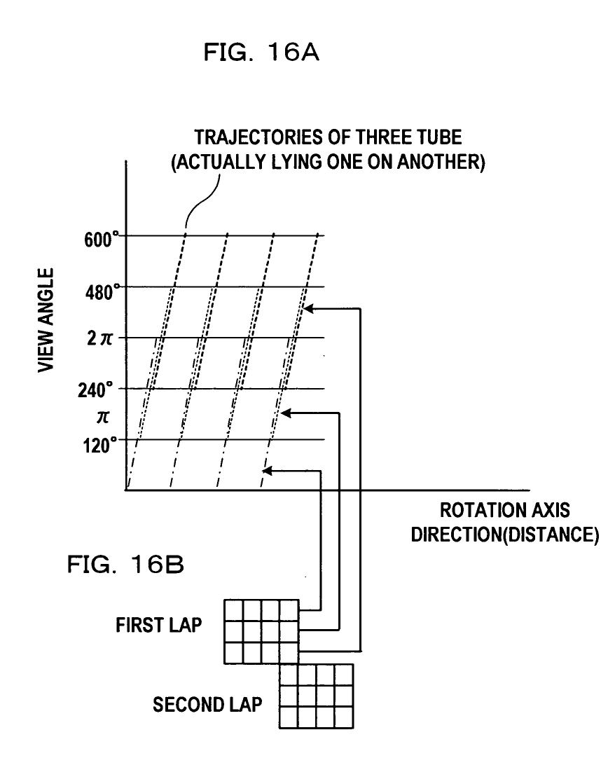

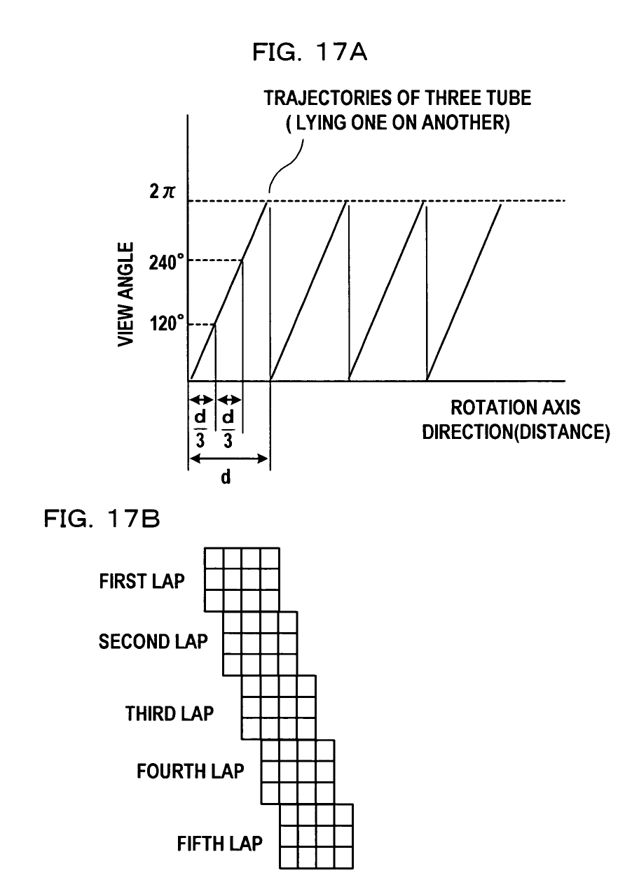

FIGS. 16A, 16B to 17A, 17B are diagrams showing examples which are suitable for improving the temporal resolution.

FIGS. 16A, 16B are diagrams showing when three multi-slice detectors 31A-31C are disposed in alignment to the rotation axis direction. As shown in FIGS. 16A, 16B, there is a measurement diagram created by three pairs of X-ray tubes 21A-21C and multi-slice detectors 31A-31C with the offset coefficient N=1 and the helical pitch P=3, wherein the three pairs of X-ray tubes 21A-21C and multi-slice detectors 31A-31C are disposed at intervals of 120° in the rotating direction. In this measurements, trajectories of the three pairs of X-ray tubes 21A-21C and multi-slice detectors 31A-31C completely match (in the figure, they are separately shown for facilitating the understanding).

The helical pitch P when the trajectories of the respective multi-slice detectors completely match is calculated by the following equation:
P=3×N (3)
where N: the offset coefficient of the deviation (offset) ΔZ.

In comparison of the embodiment shown in FIGS. 16A, 16B with the conventional one-tube four-row detector CT, there are three pairs of X-ray tubes 21A-21C and multi-slice detectors 31A-31C, i.e., three times as much. Also, there are two rows in the array of multi-slice detectors 31A-31C which overlap in the first and second rounds, which is twice as much. Further, the interpolation of opposite data provides the temporal resolution twice as high. In total, when the temporal resolution is represented by the ratio to the speed per rotation of the scanner, two rows of overlapping arrays x three tubes x opposite data (two)=12, thus improving by a factor of 12 as compared with the conventional one.

Similarly, FIGS. 17A, 17B are diagrams showing when the offset coefficient of the deviation (offset) ΔZ in the rotation axis direction is set to N=1/3. Calculating this condition by Equation (3), the trajectories of the respective multi-slice detectors match when the helical pitch P is one. In the figure, the trajectories are drawn as a single line. Then, as shown in FIG. 17, there are four rows of the arrays of the multi-slice detectors 31A-31C of FIGS. 16A, 16B which overlap in the first and fourth rounds, which are four times as much, and the remaining conditions are the same, so that the temporal resolution is calculated as four rows of overlapping arrays x three tubes x opposite data (two)=24, thus improving by a factor of 24 as compared with before.

Also, in consideration of the interpolation of opposite data shown in FIGS. 9A, 9B and FIGS. 10A, 10B, the helical trajectories match in all the rows in FIGS. 16A, 16B and FIGS. 17A, 17B, so that the temporal resolution is improved.

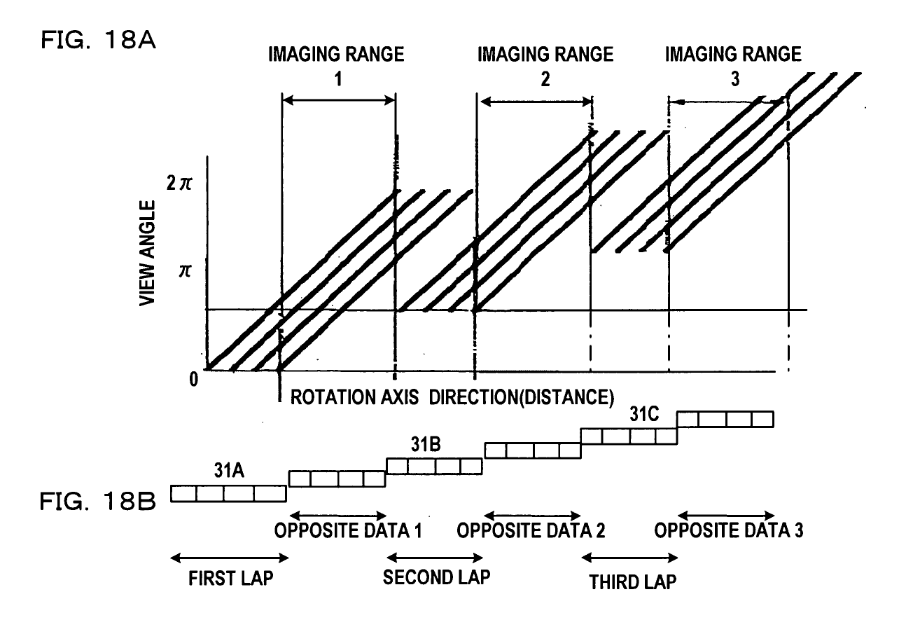

FIGS. 18A, 18B are diagrams showing an exemplary imaging operation with three pairs of X-ray tubes 21A-21C and multi-slice detectors 31A-31C. As shown in FIGS. 18A, 18B, projection data in imaging ranges 1, 2 and 3 are simultaneously measured by three pairs of X-ray tubes 21A-21C and multi-slice detectors 31A-31C. In the imaging ranges 1-3 shown in FIG. 18A, 18B, the projection data can be derived from opposite data 1-3 of measured values captured by the three multi-slice detectors 31A-31C. According to the method of FIGS. 18A, 18B, for example, by simultaneously measuring a cervical part of the subject 16 in the imaging range 1, the internal tissue within the brain in the imaging range 2, and brain blood vessels in the imaging range 3, an effective tomographic image can be captured.

The foregoing imaging operation with the three pairs of X-ray tubes 21A-21C and multi-slice detectors 31A-31C will be described with reference to FIG. 3. First, the operator selects imaging conditions in accordance with the purpose of diagnose and observation through the data input unit 41. Then, in this embodiment, the operator can select one from three imaging modes: a high speed imaging mode, a rotation axis direction resolution preference mode, and a temporal resolution preference mode on the data input unit 41, making use of the aforementioned features (high speed, high resolution). Further, through the data input unit 41, the operator enters measurement parameters related to the measurement into the host computer 11, including imaging ranges, and the geometry (imaging geometric system) of sets of the X-ray tubes 21 and multi-slice detectors 31.

Then, the host computer 11 sets parameters in the offset control unit 63, subject table control unit 61, and measurement control unit 51 in accordance with the conditions selected through the data input unit 41. After the respective units are ready for imaging, including an offset adjusting operation prior to the rotation of the scanner 12, instructed by the offset control unit 63, the host computer 11 is notified from the respective control units that imaging can be made. As the start of imaging is instructed, X-rays are substantially simultaneously emitted from the three X-ray tubes 21A-21C in accordance with the indicated X-ray conditions. Since a scan over one rotation (360°) can be made only by rotating the scanner 12 by 120°, an effective scanning time (temporal resolution) is reduced by a factor of three, leading to an improvement on the temporal resolution.

Also, with the provision of a mechanism which can move the three pairs of imaging geometric systems in the rotation axis direction, an imaging range and a temporal resolution can be appropriately selected depending on a particular site to be imaged, and a rapid diagnosis and the like can be made on a region of interest of the operator.

Therefore, when the rotational speed of the rotary disc 49 shown in FIGS. 2A-2C is set to 0.6 seconds, the scanning time is 0.2 seconds in this embodiment, thus realizing a high speed multi-slice X-ray CT device. Also, the 0.2-second scan can be realized without relying on dynamic scanning or helical scanning.

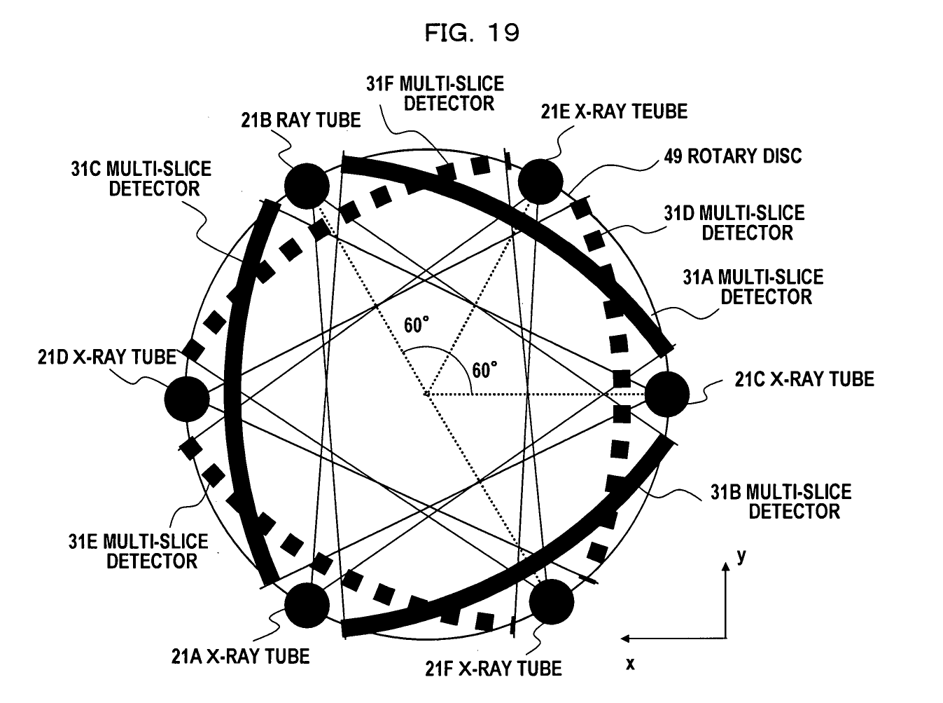

Next, FIG. 19 is a diagram showing an embodiment which employs six X-ray tubes. As shown in FIG. 19, when a rotation phase difference is set to 60° for X-ray tubes 21A-21F and multi-slice detectors 31A-31F mounted on the rotary disc 48, the operation for 360° can be performed only with a rotation over 60° of rotating angle, thus making it possible to realize high speed helical scanning.

Two sets (each including three pairs) are disposed at intervals of 120° of rotating angle. A first group comprises three pairs of X-ray tubes 21A-21C and multi-slice detectors 31A-31C, and a second group comprises three pairs of X-ray tubes 21D-21F and multi-slice detectors 31D-31F. Therefore, the condition is that the first group is offset from the second group in the rotation axis direction, such that X-rays radiated from the X-ray tubes 21A-21F do not interfere with one another.

Next, the data processing in the foregoing embodiment will be described in detail.

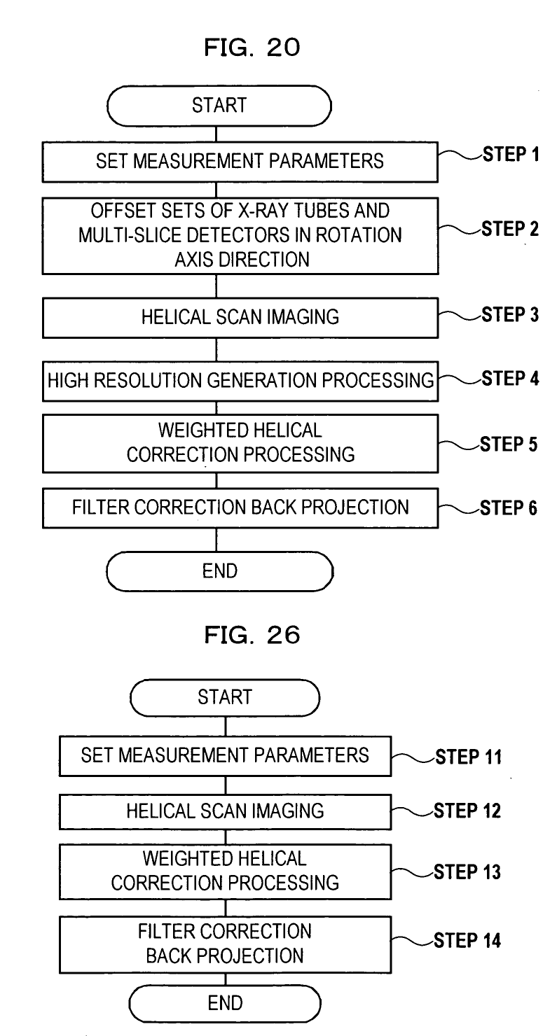

FIG. 20 is a diagram illustrating a processing flow of the multi-slice X-ray CT device. As illustrated in FIG. 20, description will be made herein on a method of making a measurement with the same trajectory, and generating a high resolution image based on the measurement. Then, as illustrated in FIG. 20, setting of measurement parameters (step 1), helical scan imaging (step 3), weighted helical correction processing (step 5), and filter correction back projection processing (step 6) show a conventional tomographic image creating method. This embodiment adds a (offset) procedure (step 2) for offsetting sets of X-ray tubes 20 and multi-slice detectors 30 in the rotation axis direction (step 2), and high resolution generation processing (step 4).

For generating high resolution data, measurement parameters related to the measurement such as a moving speed of the subject table 13, tube currents of the respective X-ray tubes 21A-21C, and the geometry of the sets of X-ray tubes 21A-21C and multi-slice detectors 31A-31C (the distance between the X-ray tubes 21A-21C and multi-slice detectors 31A-31C, and the distance between the X-ray tubes 21A-21C and the center of rotation) are entered into the host computer 11 from the data input unit 41 (step 1).

Further, as measurement parameters to be entered, an X-ray irradiation field is limited using the slice collimators 48A-48C in accordance with a region of interest of the subject 16 in the rotation axis direction as well as in the direction in which the X-ray tubes 21A-21C are rotated (step 1).

In steps 2-6 of the processing flow for the multi-slice X-ray CT device illustrated in FIG. 20, each processing time increases corresponding to the size of an imaging range. The setting of the region of interest of the subject 16, which is defined by setting the measurement parameters, results in a reduction in testing time, so that burdens on the subject 16 can be reduced.

Based on the entered measurement parameters, the sets of X-ray tubes 21A-21C and multi-slice detectors 31A-31C installed in the scanner are shifted (step 2) in the rotation axis direction for helical scan imaging (step 3), such that the respective X-ray tubes 21A-21C measure along the same trajectory.

Next, high resolution projection data generation processing is performed for generating single high resolution projection data from a plurality of projection data captured by the imaging (step 4). Also, the weighted helical correction processing is performed on the generated high resolution projection data to generate corrected projection data (step 5). Then, the generated corrected projection data is processed by the filter correction back projection to create a high resolution image (step 6).

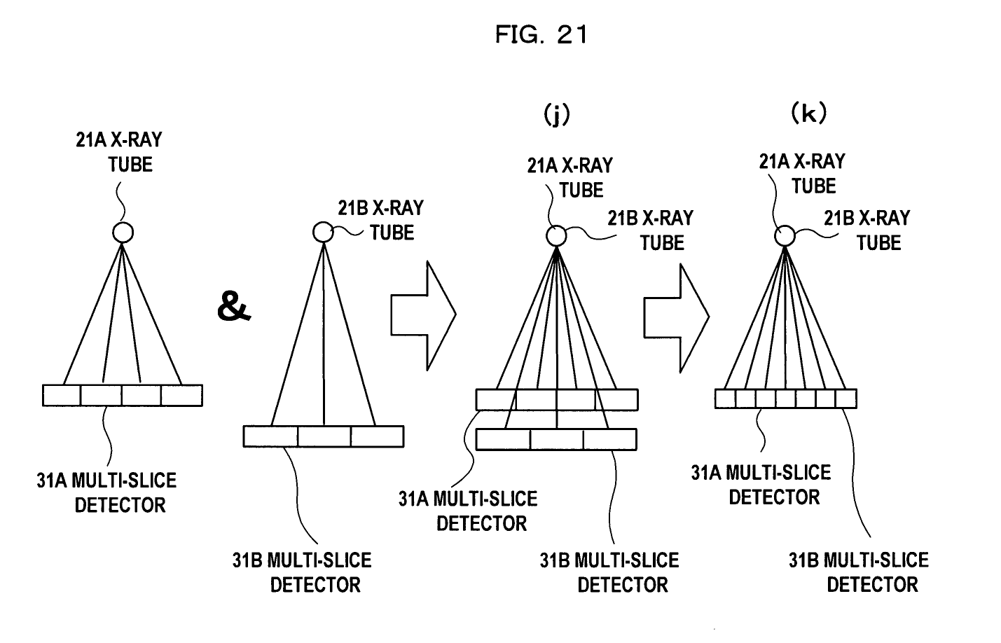

FIG. 21 is a diagram for describing contents of the high resolution generation processing shown at step 4 in FIG. 20. As illustrated in FIG. 21, there are exemplary diagrams when the imaging is performed with different geometries for the respective sets of X-ray tubes 21A, 21B and multi-slice detectors 31A, 31B. In FIG. 21, the positions at which the respective X-ray tubes 21A and 21B are mounted are adjusted such that X-rays from the respective X-ray tubes 21A and 21B pass different paths from each other.

Also, when a plurality of multi-row multi-slice detectors 31A-31B are disposed at equal intervals in the rotation axis direction, there is a method in which projection data of the multi-slice detectors 31A-31B include projection data of a plurality of rows which differ in thickness from one another. According to this method, projection data of rows having a smaller thickness can be acquired from the projection data of a plurality of rows different in thickness by a calculation, as compared with the projection data before the calculation.

The adjustments of these geometries, or a plurality of rows different in thickness, will not contribute to an improvement on the resolution of the resulting projection data, but the paths of the X-ray beams are different between the projection data at the same phase in the projection data of the different X-ray tubes 21A-21C, mutually increase the density of data sampling, so that a higher resolution can be realized even when the half reconstruction is used.

In FIG. 21, the multi-slice detector 31A has an array comprised of four uniformly sized rows, while the multi-slice detector 31B is comprised of thee rows of uniformly sized elements with a pitch width P equal to that of the X-ray tube 31A. The imaging can be made with beam paths as illustrated in FIG. 21(j) by measuring along the same trajectory with the X-ray tubes 21A, 21B and multi-slice detectors 31A, 31B. As a result, as illustrated in FIG. 21(k), the measurement made by the multi-slice detectors 31A and 31B provides the number of slices equal to seven rows, realizing a high density multi-slice X-ray CT device.

Further, by shifting the multi-slice detectors 31A and 31B in the vertical direction in FIG. 21, i.e., the rotating direction, a high density multi-slice X-ray CT device with a fine pitch is realized.

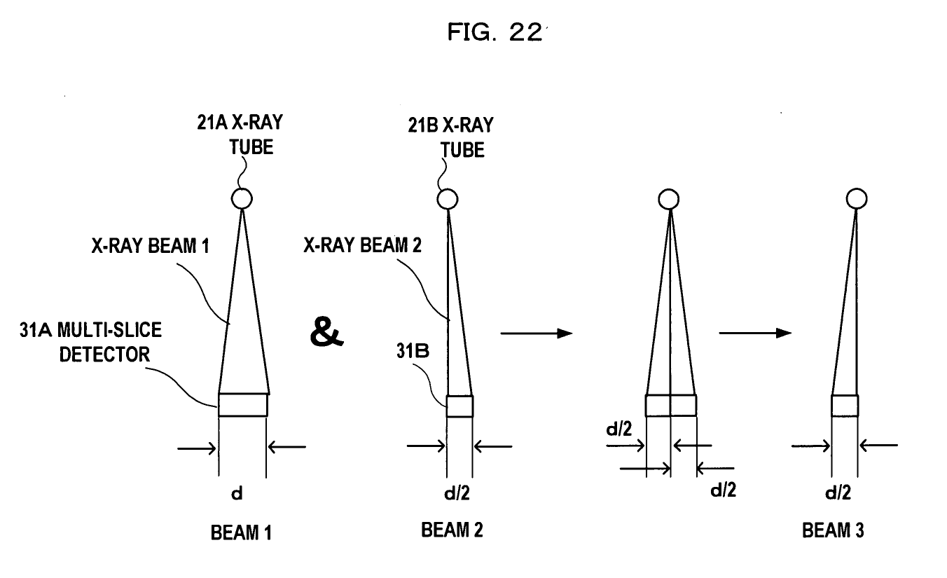

FIG. 22 is a diagram for describing an example of high resolution generation processing shown at step 4 in FIG. 20. As illustrated in FIG. 22, this is an example of calculating an X-ray beam 3 having a width of d/2, different from an X-ray beam 2, from an X-ray beam 1 having a width of d emitted from the X-ray tube 21A and the X-ray beam 2 having a width of 2/d emitted from the X-ray tube 21B. In this example, the X-ray beam 2 is irradiated to one half of the multi-slice detector 31B on one side. In consideration of the thus irradiated X-ray beam, it is apparent that projection data of the X-ray beam 3 can be accurately calculated by differentiating projection data of the X-ray beam 2 from the X-ray beam 1.

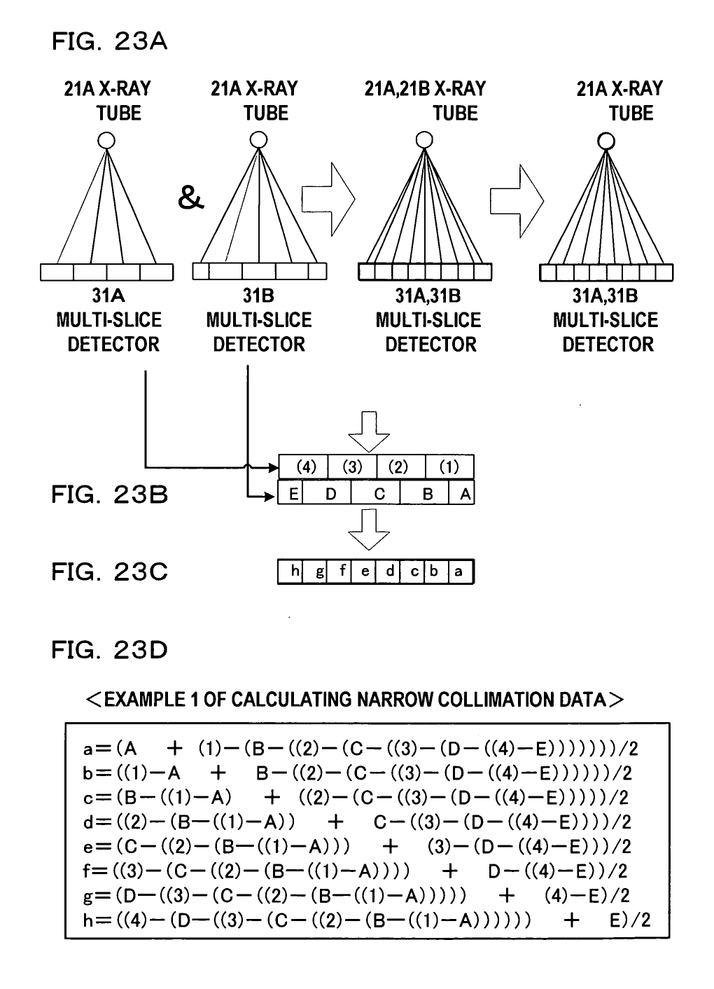

FIGS. 23A-23D are exemplary diagrams illustrating elements of different sizes arranged for each set of X-ray tubes 21A-21B and multi-slice detectors 31A-31B. As illustrated in FIGS. 23A-23D, in this event, each of the multi-slice detectors 31A-31B are provided with elements having different element widths, and the density of projection data is increased making use of the difference.

FIG. 23B shows sampling positions by both the multi-slice detectors 31A and 31B (multi-slice detector X-ray tube 31A: the first to fourth rows are (1)-(4), and multi-slice detector X-ray tube 31B: the first to fifth rows are A-E) when a measurement is made along the same path. FIG. 23C shows sampling positions (a-h) derived from the processing for increasing the resolution. Thus, from FIGS. 23A and 23B, a is equal to A, and b can be calculated by subtracting A from (1). Similarly, c can be calculated by subtracting b from B.

In this event, if an error such as noise is included in the projection data captured by the detector, the influence of the error such as noise can accumulate as the calculation is advanced (as the position is closer to the opposite end). Therefore, as shown in equations described in FIG. 23D, with exemplary calculations of a-h using (1)-(4) and A-E, similar calculations may be made from the opposite end as well, and the results derived from both may be averaged to correct for the influence of the error and acquire satisfactory high resolution projection data.

Thus, as illustrated in FIGS. 23A-23D, X-ray beams having a narrow width are disposed at the ends, and differential calculations are made sequentially from one end, thereby making it possible to increase the resolution to the opposite end.

In the example shown herein, high resolution data are calculated from two narrow projection data (high resolution data) by calculations, but ideally, a larger number of narrow projection data are preferably provided and are relied on to make a correction.

It is therefore apparent that according to this embodiment, error-free, highly accurate, and high resolution tomographic images can be generated without using such processing which deteriorates projection data through interpolation or the like. Also, with the method illustrated in FIGS. 23A-23D, a three-dimensional tomographic device is realized having means for generating high resolution projection data from projection data captured by imaging.

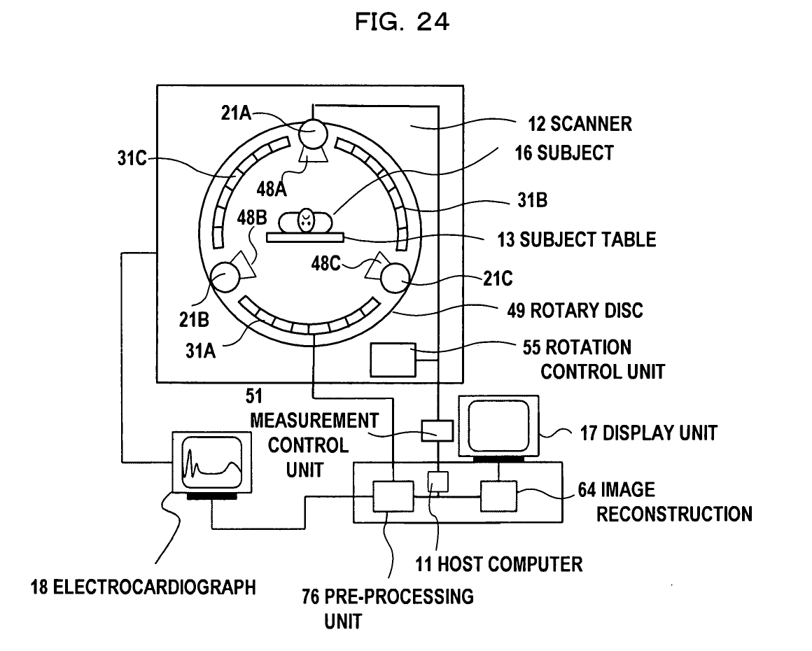

Next, FIG. 24 is a diagram illustrating the configuration of the multi-slice X-ray CT device according to this embodiment. As illustrated in FIG. 24, the multi-slice X-ray CT device comprises the scanner 12 for irradiating X-rays and detecting X-rays; the pre-processing unit 76 for creating projection data from measured data detected by the multi-slice detectors 31A, 31B, 31C; the image processing unit 78 for processing the projection data into a CT image signal; and the image display unit 43 for outputting a CT image.

The scanner 12 is mounded with the rotary disc 49; X-ray tubes 21A, 21B, 21C mounted on the rotary disc 49; slice collimators 48A, 48B, 48C attached to the X-ray tubes 21A, 21B, 21C for controlling the direction of X-ray bundles; and the multi-slice detectors 31A, 31B, 31C mounted on the rotary disc 49. The rotary disc 49 is rotated by the rotation control unit 55, while the rotation control unit 55 is controlled by the measurement control unit 51.

The intensity of X-rays generated from the X-ray tubes 21A, 21B, 21C is controlled by the measurement control unit 51. The measurement control unit 51 in turn is operated by the host computer 11. Further, the pre-processing unit 76 is connected to an electrocardiograph 18 for capturing an electrocardiogram of the subject 16.

Then, transmission data detected by the multi-slice detectors 31A, 31B, 31C is transferred to the pre-processing unit 76 which forms projection data with less artifact from the electrocardiogram of the subject 16 measured by the electrocardiograph 18, and imaging conditions provided from the measurement control unit 51. The resulting projection data is reconstructed to a tomographic image of the subject 16 by the image processing unit 78, for display on the image display unit 43.

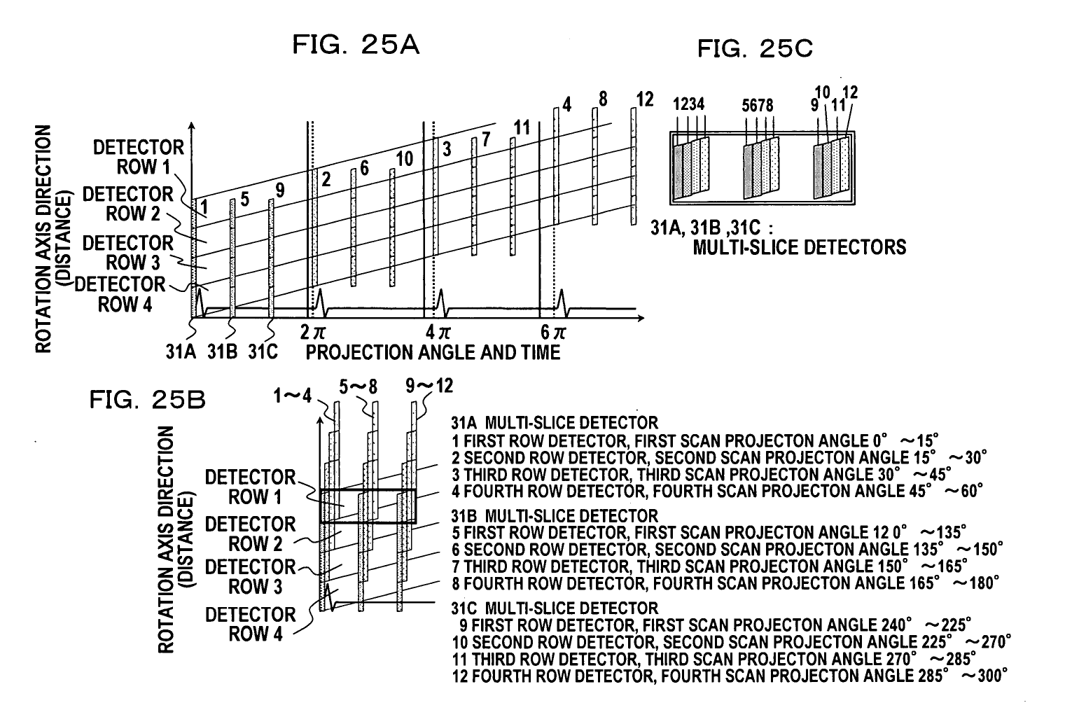

FIGS. 25A-25C are diagrams illustrating an image reconstruction method for reconstructing an image from projection data of the multi-slice X-ray CT device. As illustrated in FIG. 25A, the vertical axis represents the distance in the rotation axis direction, while the horizontal axis represents a projection angle and time. Also, an ECG signal is shown below the horizontal axis to indicate the heart phase in the rotating angle direction. Then, imaging conditions are assumed to define the helical pitch equal to one, the number of rows equal to four in the multi-slice detectors 31A-31C, and the period of the heard phase, converted to the angle, equal to 2π×(25/24) with respect to the scan period of 2π. Here, the helical pitch is defined to be the ratio to a detector element arrangement pitch in the rotation axis direction.

FIG. 25B is a diagram showing a collection of the projection data 1-12 in FIG. 25A.

A rectangle in FIG. 25B represents the projection data of detector elements 1-4 in four rows at the center of rotation when a helical scan is performed, indicating projection data which are equal in heart phase. Also, projection data after collection at the first scanning is shown for facilitating the understanding of a method of collecting divided projection data.

Next, a rectangle partitioned into 12 pieces in FIG. 25C is an enlarged view of the collected projection data, and each of the partitioned areas indicates each of collected divided projection data (1)-(4), respectively, representing detector data, the number of scans from the start of scanning, and a range of projection angle of the respective divided projection data. In this way, projection data which are different in the number of scans and equal in heart phase are collected (in the case of this figure, since the half reconstruction is performed, collected is projection data over approximately 240° which is 180°+fan beam angle) for reconstructing an image.

In FIGS. 25A-25C, the projection data over approximately 240°, which is a rotation angle required for 180° reconstruction, is created by coupling divided projection data which are provided from the respective multi-slice detectors 31A-31C that have four rows of slices.

Three rectangles positioned on the same scan represent projection data 1-12 which are generated from the respective sets of X-ray tubes 21A-21C and multi-slice detectors 31A-31C at the same time. Then, for processing the projection data 1-12 to reconstruct an image, the projection data are integrated for each of the multi-slice detectors 31A-31C, as shown in FIG. 25B. These projection data are projection data which have the projection angles that are shifted by 120°.

Also, in intervals of the respective projection data, i.e., in ranges of 60° to 120° and 180° to 240°, the opposite data derived by the method described in FIGS. 9A, 9B and FIGS. 10A, 10B are interpolated to reconstruct an image.

In the multi-slice X-ray CT device, an image can be reconstructed from projection data of three tubes placed at intervals of 60° as illustrated in FIG. 25C. Therefore, with a three-tube type multi-slice X-ray CT device, projection data over an angle of 60°, for ⅙ scan, per tube is required for reconstructing an image at an arbitrary slice position.

In FIGS. 25A-25C, the projection data over an angle of 60° required for reconstructing an image are created by coupling divided projection data respectively derived from the multi-slice detectors 31A-31C which have four rows of slices. Specifically, the projection angle of the divided projection data per multi-slice detector 31A is an angle over which the rotary disc 49 is rotated in (60°/360°)×(¼) scans. In FIGS. 25A-25C, therefore, a temporal resolution 1/24 as low as the scanning period is achieved.

Otherwise, an attempt has been made to improve an effective temporal resolution by establishing the synchronization with an electrocardiogram. This has been realized by creating a tomographic image with multiple slices, wherein a theoretical temporal resolution can be improved to approximately one fifth by measuring the same cardiac cycle (heart phase) at the same slice position, for example, a diastolic phase of the heart with each detector array. The theoretical temporal resolution can reach up to one quarter of half scan at maximum in a four-row multi-slice, and conditions such as movements of the subject table, a scanning time, and the like are set such that a view range required for reconstruction (180°+fan angle for half scan) is divided into four segments, each of which can be measured by a different row.

In a general heart CT test, for reducing motion artifact due to the beat of the heart, an electrocardiographic wave is added to scanned data to collect the projection data, and projection data at the same heart phase over a projection angle required for reconstructing an image are collected from a plurality of scanned data, to reconstruct the image. Also, the scan period and the amount of movement of the subject table are adjusted depending on the heart rate of the subject. Further, the projection data is efficiently collected by establishing the synchronization between the scanner rotation period and the cardiac cycle.

Then, actions taken for observing how the heart is pulsating, involve dividing one heartbeat into several heart phases, combining divided projection data substantially equal in divided heart phase to create projection data which is reconstructed into an image, and sequentially displaying produced tomographic images of the heart or three-dimensional tomographic images produced from a plurality of tomographic images of the heart in the order of the heart phase.

In the current X-ray CT device which provides a scan speed of approximately one second, X-rays are intermittently emitted based on electrocardiographic information of a patient to measure projection data which are at the same heart phase and at different projection angles for one scan. Then, this measured data is used to reconstruct an image. This is generally referred to as an electrocardiographic gate function or an ECG (ECG: Electro Cardio Graph) trigger. There has also been proposed a method which captures (images) projection data without synchronization to the cardiac cycle, and combines those projection data which are at the same heart phase, after the projection data have been captured, to reconstruct an image. This method is generally referred to as an ECG gate imaging.

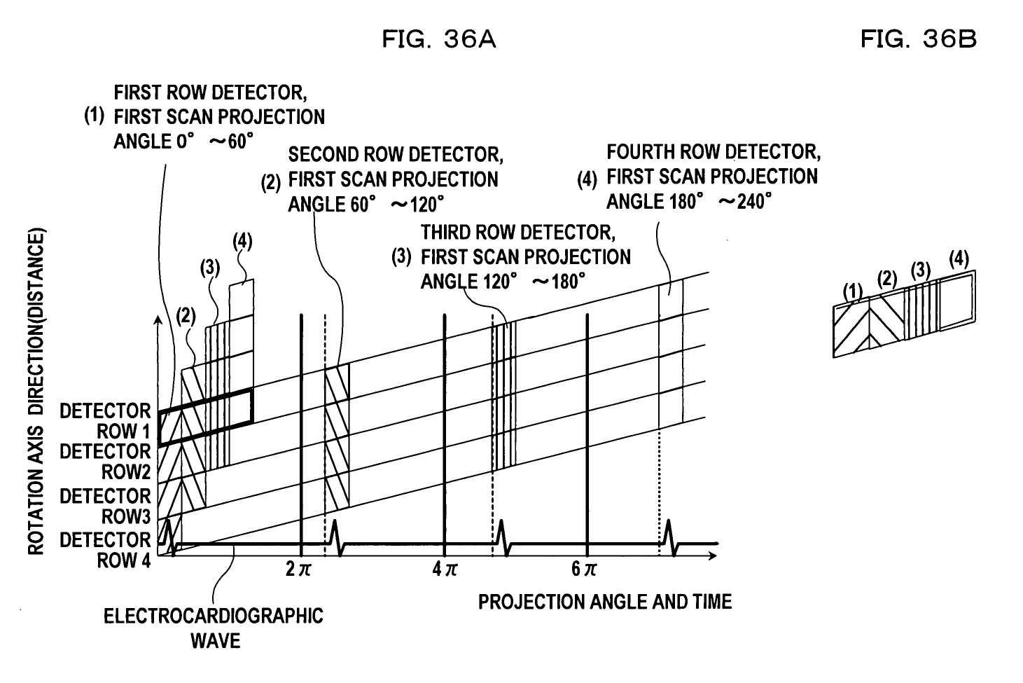

FIGS. 36A, 36B illustrate a combination of a conventional X-ray CT device and an ECG gate scan. As illustrated in FIGS. 36A, 36B, the vertical axis represents the distance in the rotation axis direction, while the horizontal axis represents the projection angle and time. Below the horizontal axis, an ECG signal is also shown to indicate the position of heart beat. Imaging conditions are assumed to define the helical pitch equal to one, the number of detector rows equal to four, the scanning cycle equal to 0.6 sec, and the cardiac cycle equal to 0.7 sec. Here, the helical pitch is defined to be the ratio to a detector element arrangement pitch in the rotation axis Z-direction.

Then, rectangles in FIG. 36A represent projection data of detector rows 1-4 at the center of rotation when the helical scan is performed, showing projection data which are at the same heart phase. Also, projection data after collection in the first scan (cycle) are shown here for facilitating the understanding of a method of collecting divided projection data.

Next, a rectangular partitioned into four pieces in FIG. 36B is an enlarged view of the projection data after collection, where the respective partitioned areas represent respective collected divided projection data (1)-(4), which show detector data, the number of scans from the start of scanning, and a projection angle range of the respective divided projection data. In this way, projection data which are different in the number of scans but are equal in heart phase are collected (in the case of FIGS. 36A, 36B, since the half reconstruction is performed, collected is projection data over approximately 240° which is 180°+fan beam angle) for reconstructing an image.

The 180° reconstruction method requires projection data for approximately 2/3 scans (180°+fan angle) in order to provide a reconstructed image at an arbitrary slice position.

When electrocardiograph synchronized reconstruction is performed by a multi-slice X-ray CT device comprised of a pair of X-ray tube 21A and multi-slice detector 31A, projection data different in cardiac cycle are combined.

Here, in the electrocardiograph synchronized reconstruction by three pairs of X-ray tubes 21A-21C and multi-slice detectors 31A-31C as in this embodiment, since an image is reconstructed from projection data measured at the same time, a resulting tomographic image excels in the image quality.

The temporal resolution in imaging with a scan cycle being S [sec] and the multi-slice detector 31A having L rows, can be calculated from an equation Sx(⅙)×(1/L). As a result, since the resulting temporal resolution is four times higher as compared with the conventional method (FIGS. 36A, 36B), a tomographic image of an overall heart, i.e., a three-dimensional tomographic image can be produced.

Also, a three-dimensional moving image (tomographic images) of a continuously beating heart, i.e., a smooth four-dimensional tomographic image can be produced by creating a plurality of tomographic images of the heart at heart phases at arbitrary time intervals, and collecting the created tomographic images of the heart for each heart phase in the rotation axis direction a plurality of times to display three-dimensional tomographic images at the heart phases at arbitrary time intervals in the order of the heart phases on the image display unit 43.

When such a projection data collecting method is used, it is possible to adjust the scan cycle, the width of divided projection data, and the number of divided projection data to synchronize the measurement with the heart phase.

When divided projection data equal in heart phase are collected from projection data of the respective multi-slice detectors 31A-31C, the pre-processing unit 76 can form projection data which are equal to an arbitrary heart phase indicated by the operator and extend over a projection angle range required for reconstructing an image by adjusting the first projection angle of the divided projection data.

Then, the image processing unit 78 can produce tomographic images of the heart at arbitrary slice positions, respectively, for a plurality of projection data provided from the pre-processing unit 76.

Further, when an attempt is made to realize a temporal resolution equivalent to the conventional method, a less number of divided data is required. As a less number of divided data is to be collected, irregular heart phases will be less likely to exert influences, thus improving tomographic images of the heart in image quality. Also, as a less number of divided projection data is to be combined, it is possible to reduce artifact caused by discontinuity of projection data at junctions of divided projection data.

FIG. 26 is a diagram illustrating a processing flow in another embodiment of the multi-tube multi-slice X-ray CT device. As illustrated in FIG. 26, described herein is a method of making measurements along the same trajectory and generating a high resolution image based on the measurements. Then, as illustrated in FIG. 26, a tomographic image of the subject 16 is created by a procedure which includes designing of measurement parameters (step 11), helical scan imaging (step 12), weighted helical correction processing (step 13), and filter correction back projection processing (step 14).

For generating high resolution data, measurement parameters related to the measurement such as a moving speed of the subject table 13, tube currents of the respective X-ray tubes 21A-21C, and the geometry of the sets of X-ray tubes 21A-21C and multi-slice detectors 31A-31C (the distance between the X-ray tubes 21A-21C and multi-slice detectors 31A-31C, and the distance between the X-ray tubes 21A-21C and the center of rotation) are entered into the host computer 11 from the data input unit 41 (step 11).

Further, as the entered measurement parameters, conditions for limiting an X-ray irradiation field in the rotation axis direction and in the direction in which the X-ray tubes 21A-21C are rotated are set in accordance with a region of interest of the subject 16 (step 11).

In steps 2-4 of the processing flow for the multi-slice X-ray CT device illustrated in FIG. 26, each processing time increases corresponding to the size of an imaging range. As such, the setting of the region of interest of the subject 16, defined by setting the measurement parameters, results in a reduction in testing time, so that burdens on the subject 16 can be reduced.

Based on the entered measurement parameters, helical scan imaging is performed with the sets of X-ray tubes 21A-21C and multi-slice detectors 31A-31C mounted in the scanner (step 12).

Next, a plurality of projection data captured by the imaging are subjected to the weighted helical correction processing to generate corrected projection data (step 13). Then, the generated corrected projection data is processed by the filter correction back projection to create a high resolution image (step 14).

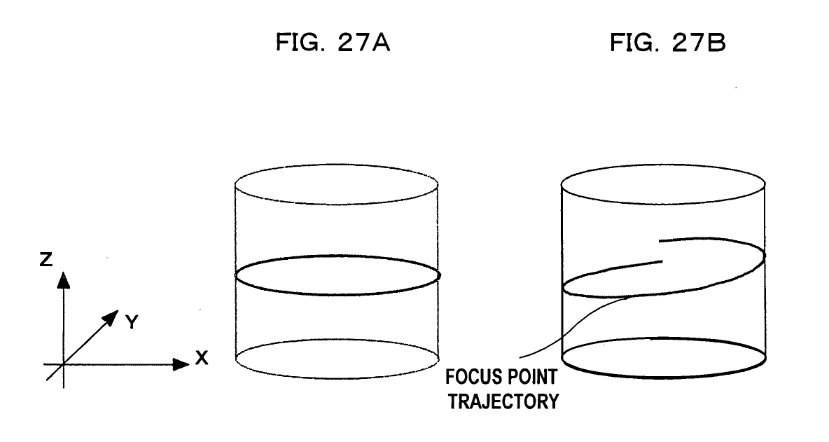

FIGS. 27A, 27B are diagrams showing a circular trajectory scan and a helical trajectory scan. As shown in FIG. 27A, a filter correction back projection method should be applied to projection data imaged along a circular trajectory, i.e., generated from X-rays irradiated from X-ray tubes which are rotated above an image to be reconstructed, so that if this method is applied to projection data generated by a helical trajectory scan as shown in FIG. 27B, large distortion will occur. For this reason, when imaging is performed along a helical trajectory as shown in FIG. 27B, the helical trajectory is interpolated to a circular trajectory for reconstruction based on the circular trajectory.

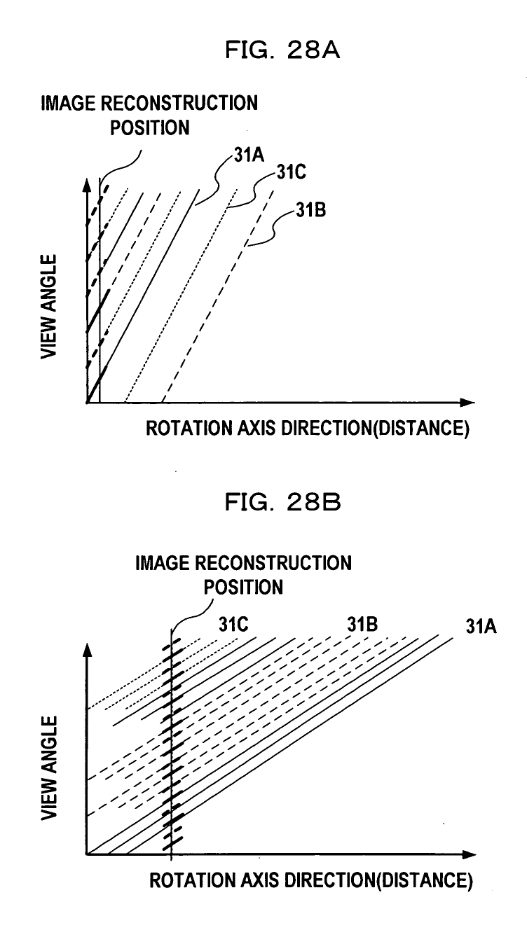

Next, FIGS. 28A, 28B show diagrams of measured trajectories when the reconstruction is made by interpolating a helical trajectory into a circular trajectory. In FIGS. 28A, 28B, solid lines indicate actually measured real data trajectories, and broken lines indicate trajectories of opposite data which are positioned diametrically opposite to the real data trajectories. Also, as shown in FIGS. 28A, 28B, when a helical trajectory is interpolated into a circular trajectory for reconstruction, a weighting function which substitutes real data for opposite data may be used to maintain continuity of the phase (view) at a reconstruction position even in a shorter view range (per row). In addition, the opposite data may be virtually created from the real data.

Then, FIG. 28A is a diagram showing the trajectories of projection data measured by multi-slice detectors 31A-31C (pitch 6) which satisfy the condition for interpolating a helical trajectory into a circular trajectory, and each have one row. Also, in FIG. 28A, continuous interpolation data can be created for 360° (180°) including opposite data.

Further, FIG. 28B is a diagram showing the trajectories of projection data when a measurement is made by multi-slice detectors 31A-31C (pitch 18) having three rows.

Here, an algorithm used in the case of FIGS. 28A, 28B is a weighted helical correction reconstruction (step 13). Therefore, this embodiment is characterized in that an image can be created by use of less number of measured data from the fact that measured data do not match interpolated data at opposite positions.

Stated another way, the imaging is performed under condition that the reconstruction is possible even if no projection data exist at opposite positions (the reconstruction can be accomplished with one half of a normal view size) by substituting real data for projection data at opposite positions. This means that the temporal resolution is further improved when the imaging is performed under condition that no projection data exists at an opposite position of a certain multi-slice detector and in a certain row at a reconstruction slice position.

Then, the condition for improving the temporal resolution is established when the relationship between the helical pitch P and the number L of rows per used multi-slice detector satisfies the following conditions:

(1) when the number L of detector rows is equal to or more than two per multi-slice detector:
Helical Pitch P=2×L×K (4)

(2) when the number L of detector rows is equal to or more than one per multi-slice detector:
Helical Pitch P=K(2×Q+1)≦L×K (5)
or P=2×L×K
   where:
L (number of rows per multi-slice detectors)=1, 2, 3, . . . ;
K (number of multi-slice detectors)=1, 3, 5 . . . ; and
Q (positive integer)=0, 1, 2, . . .

The foregoing conditions hold for the most ideal case, so that values approximate to those may be used.

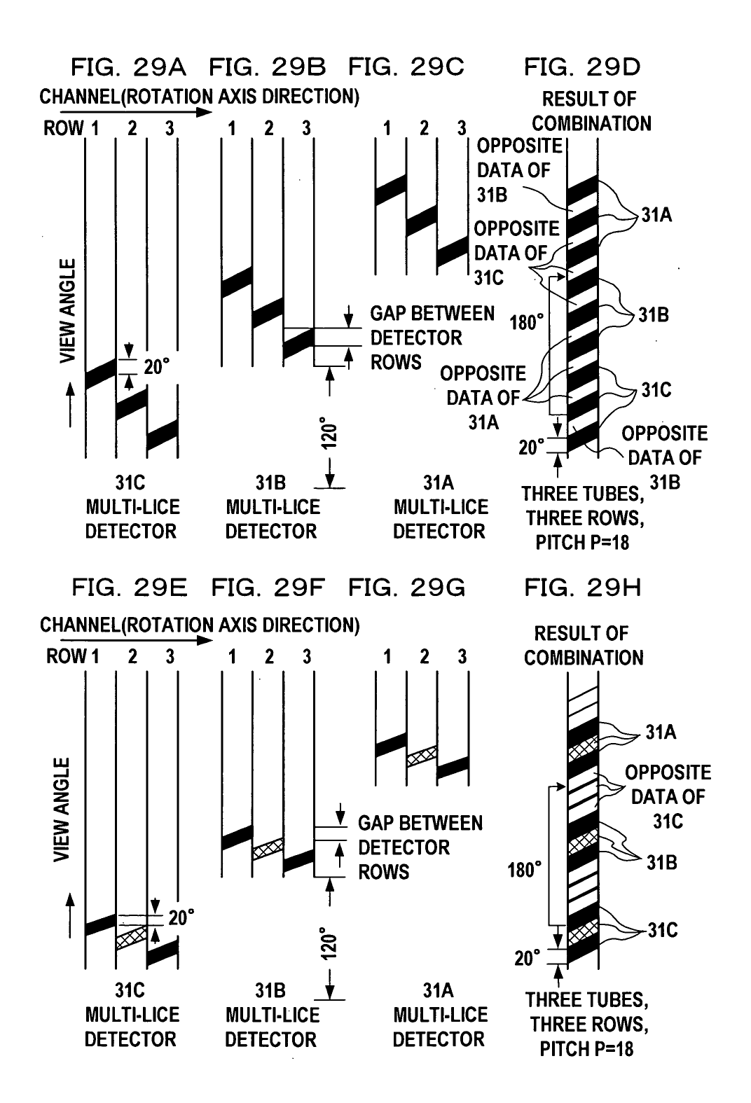

FIGS. 29A-29H are diagrams showing helical correction weights for the case of FIG. 28B. As shown in FIGS. 29A-29H, generated projection data (sinogram) is weighted by the helical correction weights to produce weighted projection data, and projection data of each row of each multi-slice detector is added to a corresponding phase to produce one corrected projection data. This corrected projection data is projected back by a filter correction to produce a reconstructed image (step 14).

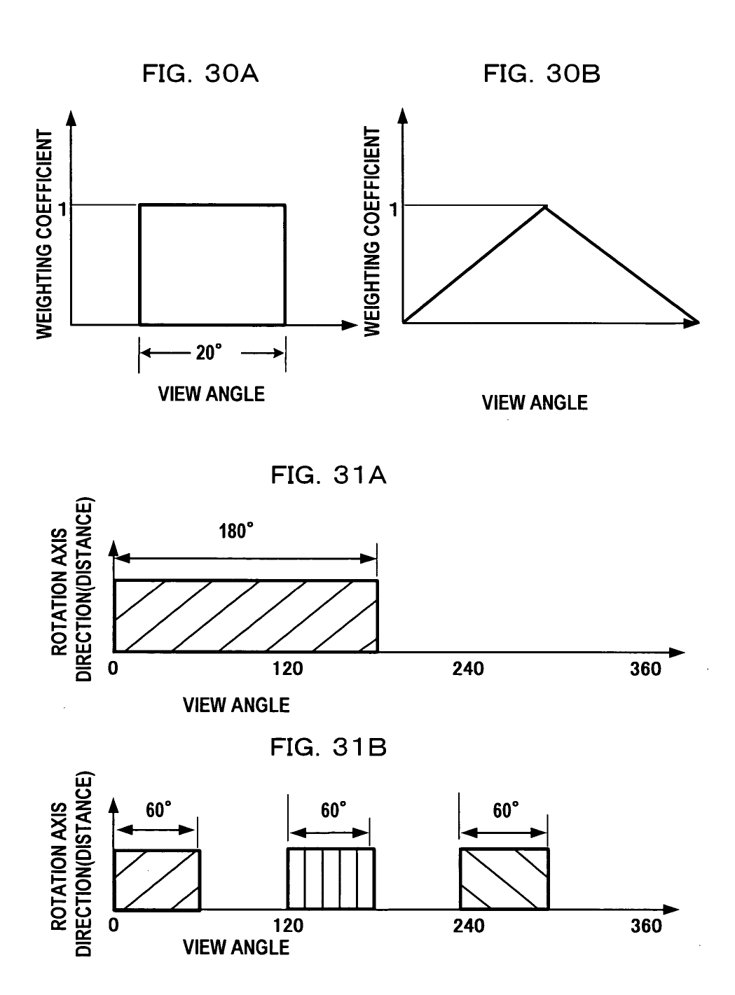

FIGS. 30A, 30B are diagrams illustrating the shapes of the respective weights. As illustrated in FIG. 30A, a weighting coefficient which changes in a step response form is used in FIGS. 29A-29H, but a weighting coefficient, the width which is extended in a applied view direction may be used as illustrated in FIG. 30B. In FIG. 30B, abrupt changes in projection data are mitigated, so that artifact due to discontinuity is reduced as compared with FIG. 30A.

FIGS. 31A, 31B show unit data when using a pair of X-ray tube and multi-slice detector, and unit data when using three pairs of X-ray tubes and multi-slice detectors (positioned at uniform angular intervals). The vertical axis in FIGS. 31A, 31B represents the distance in the rotation axis direction, and the horizontal axis represents a view angle. As shown in FIGS. 31A, 31B, consider the least amount of data (number of views) required for reconstruction when an image is created. In the following, this amount of data is referred to as the “unit data.”

As shown in FIG. 31A, the unit data in one multi-slice detector includes projection data for 180° phase (view) in the case of a parallel beam. With three multi-slice detectors, the respective multi-slice detectors differ in phase (view) from one another by 120°, resulting in discrete projection data over 60°, as shown in FIG. 31B. While this is discontinuous projection data, reconstruction is possible because continuous projection data over 180° is produced, in a manner similar to one multi-slice detector, when projection data associated with two of the three multi-slice detectors are rearranged to projection data of X-ray beams existing at opposite positions along the beam paths of the X-rays.

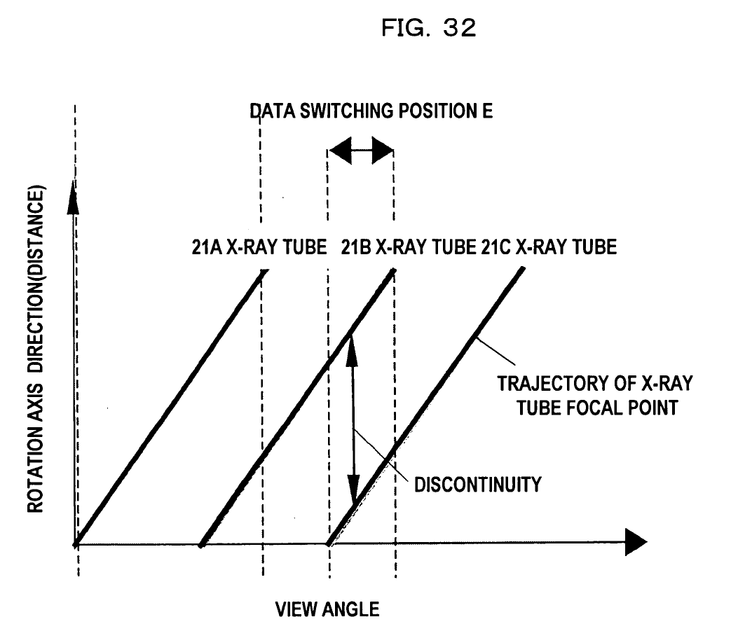

FIG. 32 is a diagram showing a trajectory of projection data by a multi-tube multi-slice X-ray CT device which has three multi-slice detectors disposed at angular intervals of 120°. As shown, FIG. 32 is a measurement diagram of projection data adjacent to one third of both ends of the projection data, measured with redundancy. Then, since the multi-slice detectors have different scan trajectories from one another, and since a data switching position E exists between the multi-slice detectors, discontinuity of projection data occurs. Due to this discontinuity of projection data, strong artifact occurs from a reconstructed image.

During weighted helical correction reconstruction, reconstruction data is created by a combination of unit data of different phases (views) at this same slice position. Therefore, the artifact can be reduced without increasing the slice thickness, similar to average addition of a plurality of images which have artifact of different phases (views), to produce an image of higher image quality.

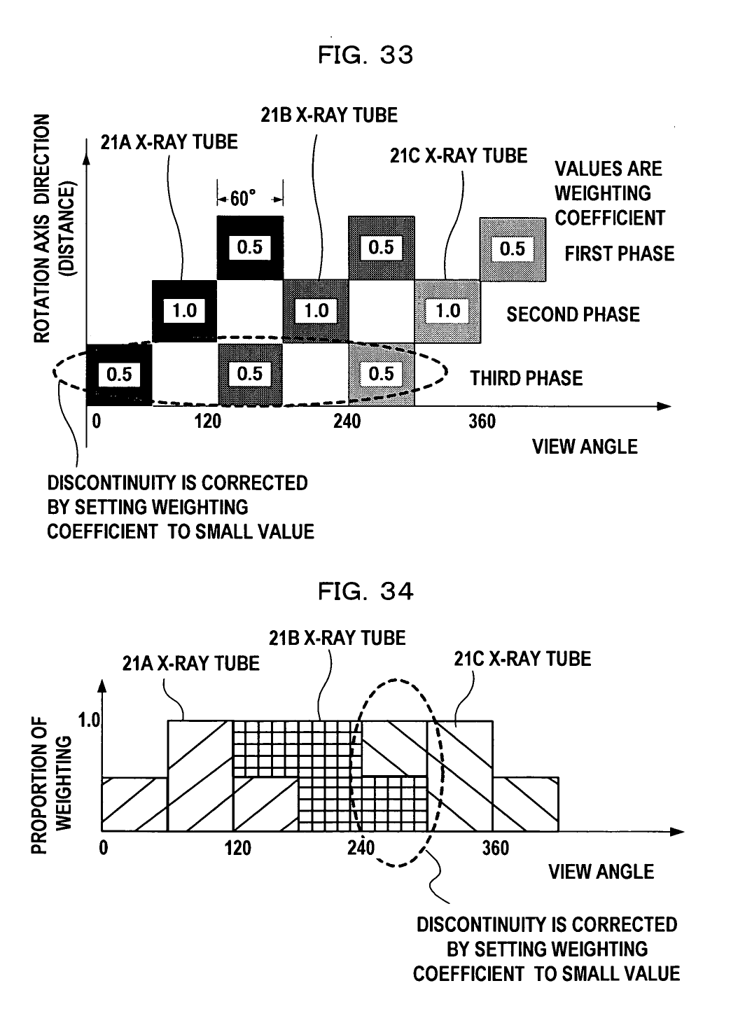

FIGS. 33, 34 show an example of a weighting function for generating a good image in the multi-tube multi-slice X-ray CT device. In FIG. 33, the vertical axis represents the distance in the rotation axis direction, while the horizontal axis represents the view (angle). Then, FIG. 33 is a diagram showing weighting (normalization) to unit data of three phases (first phase to third phase) provided by the measurement shown in FIG. 32. As shown in FIG. 33, a coefficient is multiplied for portions having redundancy to normalize them. Of course, the weighting should be such that a higher weighting coefficient is applied to a portion closer to the reconstruction slice position (second phase).

FIG. 34 is a diagram showing the proportion of weights occupied by projection data of each tube in a correction. In FIG. 34, the vertical axis represents the proportion of weights occupied by data of each multi-slice detector in corrected data resulting from the weighting, while the horizontal axis represents the view (angle). Also, as shown in FIG. 34, the proportion of weight is set to a small value of 0.5 at a position at which discontinuity occurs, and is given a relatively high value of 1.0 at the reconstruction slice position, thereby reducing the discontinuity to create a good image. In this way, for eliminating the discontinuity of projection data by the multi-tube multi-slice X-ray CT device which is equipped with three multi-slice detectors, the view (or detector column) may be weighted, as shown in FIG. 34, to generate an image of higher image quality.

As an ideal condition in this embodiment, the projection data switching position E between the respective multi-slice detectors is prevented from matching the projection data switching position of the opposite multi-slice detectors. By doing so, the discontinuity among the multi-slice detectors is corrected as well by projection data at opposite positions, thereby making it possible to generate a better image. Specifically, in the multi-tube multi-slice X-ray CT device having three multi-slice detectors disposed at intervals of 120°, when the number L of detector rows is set to a multiple Q of the number K of multi-slice detectors, as the conditions shown in Equation (6), and the helical pitch P is set to twice the number L of detector rows as the conditions shown in Equation (7), the discontinuity can be most efficiently improved:
L=K×Q (6)
P=2×L (7)
where Q (coefficient)=0, 1, 2, . . .

While the embodiment has been described for the number of X-ray tubes equal to three, similar effects can be provided as well when a multi-tube multi-slice X-ray CT device has a different number of X-ray tubes.

From the foregoing description on this embodiment, it is apparent that the object of this embodiment is achieved. While this embodiment has been described in detail and also illustrated, they are only intended for description and illustration, and the present invention is not limited to them.

Also, while this embodiment employs an X-ray based tomographic device, the present invention is not limited to this but can also be applied to a tomographic device associated with a source of radiations which have the transmittance and can be irradiated, using gamma rays and light.

Then, a single projection data can be created from a plurality of projection data captured by multiple tubes, similar to that of a one-tube type, to reconstruct an image.

Further, while each X-ray tube 21A-21C or the like is measured along the same trajectory, the present invention is not limited to this, but they may be measured along different measurement trajectories. In this case, the resolution can be increased using X-ray beams at opposite positions. Also, the respective multi-slice detectors 31A-31C or the like may be different in overall size from one another. The present invention is not either limited in the number of rows or the element size of the multi-slice detectors 31A-31C.

While the foregoing embodiment has been described for the number of X-ray tubes equal to three, similar effects can be produced as well when a multi-tube three-dimensional tomographic device has a different number of X-ray tubes.

From the foregoing description on this embodiment, it is apparent that the object of this embodiment is achieved. While this embodiment has been described in detail and also illustrated, they are only intended for description and illustration, and the present invention is not limited to them.

Also, while this embodiment employs an X-ray based tomographic device, the present invention is not limited to this but can also be applied to a tomographic device with a source of radiations which have the transmittance and can be irradiated, using gamma rays and light. Further, while the weighted helical correction reconstruction algorithm is used for a reconstruction method, the present invention is not limited to this, but any reconstruction algorithm used in a single X-ray CT device can be applied, including a three-dimensional back projection algorithm.

Then, a single projection data can be created from a plurality of projection data captured by multiple tubes, similar to that of a one-tube type, to reconstruct an image.

Further, while each X-ray tube 21A-21C or the like is measured along the same trajectory, the present invention is not limited to this, but they may be measured along different measurement trajectories. In this case, the resolution can be increased using X-ray beams at opposite positions. Also, the respective multi-slice detectors 31A-31C or the like may be different in overall size from one another. The present invention is not either limited in the number of rows or the element size of the multi-slice detectors 31A-31C.

Also, one or more multi-slice detectors may be masked to reduce the thickness of collimation, resulting in a combination of effectively narrow collimation and different collimation, to realize a higher resolution.

(Advantages of the Invention)

Description will be made on advantages provided by this embodiment.

A tomographic image of high image quality can be provided by arranging an X-ray tube and a multi-slice detector in a set, and disposing a slice collimator.

Also, three pairs of X-ray tubes and multi-slice detectors are mounted on a rotary disc, wherein the three pairs have a rotation phase difference of 120°, and are made rotatable while simultaneously holding a relative positional relationship of the imaging geometric system, thereby making it possible to realize a helical scan pitch equivalent to the number of rows substantially increased by a factor of three, only with measured data of relatively narrow cone angle, and to produce a tomographic image with a high temporal resolution and less influences of the cone angle to realize a higher image quality.

Also, a three-dimensional tomographic image of a beating heart can be smoothly created without interruption by creating a plurality of tomographic images of the heart at heart phases at arbitrary time intervals, and collecting the created tomographic images of the heart into a plurality of sets in a body axis direction for each heart phase, and a four-dimensional tomographic image can be produced in the order of the set heart phases.

Further, a high-density, high-resolution tomographic image can be produced at high speeds by adjusting the number of slices of the multi-slice detector in the rotation axis direction, and the offset of the X-ray tube and multi-slice detector.

Further, a high-resolution tomographic image can be produced by providing the three-dimensional tomographic device by providing a means for generating high-resolution projection data from projection data captured by imaging.

It is also apparent that the multi-slice detector elements, which differ for each set of X-ray tube and multi-slice detector 31, can produce a high resolution tomographic image at a high accuracy without errors by the arrayed multi-slice detectors 31.

It is also possible to form projection data with less motion artifact by collecting divided projection data equal in heart phase from the heart rate of a subject, a scan cycle of the multi-slice X-ray CT device, and the number of detector rows.

Also, the temporal resolution is improved by substituting real data for projection data at opposite positions by the multi-slice detectors.

Further, the artifact can be reduced to produce an image of higher image quality by applying a combination of unit data at different phases at the same slice position to create reconstruction data at the time the weighted helical correction reconstruction is performed.

While the foregoing description has been made on an embodiment, it is apparent that the present invention is not limited to this, but a variety of alterations and modifications can be made within the spirit of the invention and the scope of the appended claims.

1. A multi-slice X-ray CT device irradiating X-rays while rotating around the outer periphery of a subject about a body axis thereof substantially as a rotation axis and detecting X-rays which transmit the through subject, said multi-slice X-ray CT device comprising:
a plurality of pairs of X-ray sources and detector rows, wherein said X-ray sources being capable of irradiating X-rays, and said detector rows being disposed opposite to said X-ray sources across the subject and having a single row or multiple rows of detectors for detecting X-rays irradiated from said X-ray sources and transmitting through the subject to generate signals representative of the detected X-rays;
a bed for carrying the subject, movable in the rotation axis direction relatively to said plurality of pairs of X-ray sources and detector rows; and
an image reconstruction unit for processing the signals to create an image,
said multi-slice X-ray CT device wherein,
at least one of said plurality of detector rows is a multi-row detector, and said plurality of detector rows are the same as or different from one another in the width in a rotating direction, the number of rows, and the width of said detector rows.

2. A multi-slice X-ray CT device according to claim 1, wherein a mutual positional relationship among said plurality of pairs of X-ray sources and detector rows is controlled in the rotation axis direction in accordance with a desired region of interest.

3. A multi-slice X-ray CT device according to claim 1 or 2, wherein at least said X-ray sources or said detector rows are moved relative to the subject to control the mutual positional relationship among said plurality of pairs of X-ray sources and detector rows.

4. A multi-slice X-ray CT device according to any of claims 1 to 3, wherein said plurality of pairs of X-ray sources and detector rows are three pairs, a rotation phase difference between said respective pairs is 120°, and said plurality of pairs can be simultaneously rotated while the rotation phase difference is maintained.

5. A multi-slice X-ray CT device according to claim 3, wherein at least two of the number of slices in the rotation axis direction, an offset coefficient which represents a degree to which at least said X-ray sources or said detector rows are moved relatively to the subject, and a helical pitch can be set from the outside.

6. A multi-slice X-ray CT device according to any of claims 2 to 5, wherein said multi-slice X-ray CT device can be set in a high speed imaging mode, a rotation axis direction resolution preference mode, or a temporal resolution preference mode.

7. A multi-slice X-ray CT device according to any of claims 1 to 6, wherein said image reconstruction unit substitutes real data for projection data at opposite positions on the rotation phase in the signal processing.

8. A multi-slice X-ray CT device according to any of claims 1 to 6, wherein said image reconstruction unit performs the reconstruction by combining data at different rotation phases in the same slice upon weighted helical correction reconstruction in the signal processing.

9. A multi-slice X-ray CT device according to any of claims 1 to 4, wherein
for conducting high speed imaging in reconstructing an image of the region of interest, the offset coefficient, which is a degree to which at least said X-ray sources or said detector rows are moved relatively to the subject, is set to a large integer so as to expand a range to be dynamically imaged within the region of interest and simultaneously narrow down a region within the region of interest in which a high temporal resolution is desired,
said offset coefficient is set to a value less than one when a resolution in the rotation axis direction is increased in order to narrow down the range to be dynamically imaged and simultaneously increase a number into which a slice is divided on data processing, and
said offset coefficient is set to a small integer when a high temporal resolution is desired widely in the rotation axis direction so as to narrow down the range to be dynamically imaged within the region of interest and simultaneously expand the range within the region of interest in which a high temporal resolution is desired.

10. A multi-slice X-ray CT device according to any of claims 1 to 6, wherein the scan cycle and the number of rows in said detector rows are determined from measured heart rate data of the subject, divided projection data substantially equal in heart phase are collected based on the scan cycle and the number of rows in said detector rows, and a tomographic image of the heart at an arbitrary slice position is created based on the divided projection data in said image reconstruction unit.
